(19) United States
(12) Patent Application Publication
(10) Pub. No.: US 2025/0066712 A1
(43) Pub. Date: Feb. 27, 2025

(54) PROCESSES AND SYSTEMS FOR CULTURING ALGAE OR REDUCING PATHOGENIC MICROBES FROM AN AQUEOUS MEDIUM, AS WELL AS CONCENTRATES AND USES RELATED THERETO

(71) Applicant: NESTE OYJ, Espoo (FI)

(72) Inventors: Jeffrey KANEL, Porvoo (FI); David BRYANT, Porvoo (FI); Cecil CHURN, III, Porvoo (FI)

(21) Appl. No.: 18/723,835

(22) PCT Filed: Dec. 30, 2022
(86) PCT No.: PCT/FI2022/050877

§ 371 Date: Jun. 25, 2024

Related U.S. Application Data
(60) Provisional application No. 63/295,535, filed on Dec. 31, 2021.

(30) Foreign Application Priority Data
Mar. 30, 2022 (FI) 20225274

(51) Int. Cl. C12N 1/12; C12M 1/00

(52) U.S. Cl. CPC C12N 1/12; C12M 21/02; C12M 29/06

(57) ABSTRACT

Processes and systems are disclosed herein for culturing algae and/or the reduction in pathogenic microbes from shrimp, fish or other marine aquacultural process streams, e.g., waste or recycle streams, wherein the process streams are fed to an algal aquaculture pond operated at a salinity of at least about 7 wt-%, and wherein valuable nutrients are utilized by the algae.

### FIELD

The present invention relates to processes and systems for the growth of aquatic animals and/or algae, and in particular to processes and systems which utilize process streams from the growth of aquatic animals in an algal aquaculture pond. The present invention enables treating the process stream for pathogenic microbes, microbial pathogens, competitors, and/or predators, which are harmful to aquatic animals, while at the same time advantageously utilizing nutrients in the process stream for the growth of algae.

### BACKGROUND AND OBJECTS

Aquaculture is the farming of aquatic organisms, such as algae, microalgae, shrimp, fish, and shellfish. In aquaculture, saltwater and freshwater populations are cultivated under controlled conditions. The advantages of aquaculture are numerous. Seafood farmers that raise shrimp, fish, and shellfish have control of stock, quality of feed, the ability to harvest according to market demands, and the ability to process fresh seafood close to the grow-out facility and generally exercise total quality management of the process from the raw material to the dinner plate. Furthermore, aquaculture can reduce pressures on wild fisheries as long as it is performed in a sustainable manner.

There is also increasing interest in aquacultural algal farming for a plethora of sustainable activities, such as a source of renewable energy, as a source of protein, as a mode to safely and efficiently capture carbon dioxide from the atmosphere for carbon sequestration, and as a renewable source of chemical intermediates (Wijffels 2010). Algal aquaculture is a natural and sustainable management tool for biological remediation of concentrated nutrients in the wastewaters discharged from aquafarming facilities (Granada et al. 2015).

As the aquaculture industry continues to grow, so do the problems associated with aquaculture waste, particularly with the growth of aquatic animals. It is not only the sheer volume of waste material from the processing of farmed aquatic species, but also the potential for the spread of bacterial and viral pathogens through the waste that is of great concern. Control of disease is a problem further exacerbated by the relatively high stocking densities and the close proximity of one aquaculture site to another.

In an effort to maximize productivity of an aquaculture pond, such as one for growing shrimp, stocking and feeding rates are often increased. Excessive buildup of metabolic waste products and unconsumed food from feeding aquatic animals may result in low concentrations of dissolved oxygen. Deterioration of environmental conditions in the pond can lead to slow growth rates, the manifestation of disease and high mortality rates. Effluents released from ponds during water exchange and shrimp harvests may contain harmful bacteria and viruses in addition to plant nutrients, organic matter, and suspended solids. The effluent water released to coastal waters often mixes with the water intake systems for neighboring shrimp ponds, and can transfer bacterial and/or viral pathogens (Boyd 1992).

Both shellfish and fish are cultivated in intensive production systems where stress may be more prevalent and the manifestation of disease more critical. The following paragraphs cite examples for shrimp aquaculture, but similar examples may be found for shellfish and fish aquaculture systems.

With higher shrimp stocking densities, the incident of disease may be amplified, in some cases severely enough to decimate a pond's shrimp population over a short time period. In some countries a significant portion of the shrimp industry has been impacted. Accute hepatopancreatic necrosis “EMS” (early mortality syndrome) was first detected in Mexico during the 2013 shrimp production cycle and losses amounted to a decrease in industry production from 100,000 tonnes in year 2012 to less than 60,000 tonnes (Fegan, Second FAO Symposium on AHPND Bangkok, 2016).

In Mexico's Gulf of California aquaculture industry, organic debris accumulates in shrimp production ponds to such an extent that productivity may be negatively impacted after 3-4 years when remediation management strategies are limited. Currently, the only way to treat these ponds is to: 1) drain the pond, 2) allow the soil to dry, 3) disc the bottom of the pond to about 30 cms deep for exposing organic debris to solar UV and atmospheric air, 4) allow time for the accumulated debris to degrade and become oxidized, and 5) refill the ponds for restocking. North of the Tropic of Cancer, this process is performed during the “dry season” between late November to early April, as shrimp harvests conclude in late November and restocking ponds begins in March-April for an 8-month production cycle, in outdoor pond systems. This protocol occurs in the arid climate of the coastal Sonoran desert, but in many regions of the world, a “dry season” and the time between harvest and restocking do not correspond. For example, in South East Asia and other regions, calcium hydroxide is routinely added to ponds for soil pH stabilization in managing organic debris.

In 1988, Taiwan, then the top producer of industrial shrimp, lost 75 percent of its harvest to a virus called Monodon-type baculovirus (MBV). China then became the top producer, until it was hit with disease caused by hypodermal and hematopoietic virus (Lightner 2003). In 1999, Ecuador lost half of its crop to Taura syndrome (TSV) and white spot syndrome virus (WSSV). The shrimp industries of Indonesia, India, Honduras and Mexico also faced significant disease outbreaks in the 1990s and more recently as well.

Management control of disease represents a primary impediment to growth of the shrimp aquaculture industry. Historically, shrimp diseases of most importance have been: Taura syndrome virus (TSV), white spot syndrome virus (WSSV), and the necrotizing hepatopancreatitis bacterium (NHP-B).

Taura Syndrome Virus (TSV) has had a devastating economic impact on the shrimp aquaculture industry. TSV is a 30 to 32 nm, icosahedral virus particle containing positive-sense, single-stranded RNA of about 10.2 kb in length. TSV is a member of the “picornavirus superfamily,” the family Dicistroviridae and the genus Cripavirus.

White spot syndrome virus (WSSV) is the most virulent pathogen of penaeid shrimp. WSSV is an ovoidal, enveloped particle about 80-120 nm wide by 250-380 nm long with a small tail-like appendage at one end. The virion's genome consists of a single, circular, double-stranded DNA molecule of about 300 kb in length. The virus belongs to the viral family Nimaviridae and the Genus Whispovirus. There have been several publications on how to deal with WSSV, and Desal (U.S. Pat. No. 6,440,466) disclosed a composition of plant derived extracts to treat it.

White spot syndrome virus has been a principal causative agent for reduced shrimp yields worldwide. White spot syndrome is characterized by white spots that appear on shrimp flesh and cause their bodies to steadily decompose in as few as 10 days. White spot syndrome is usually accompanied by vibriosis, which is caused by Vibrio bacteria. These bacteria exist naturally in coastal waters and infect shrimp when they become stressed by problems like poor water quality, another disease or crowding. Vibrio bacteria are especially problematic: if humans eat the infected shrimp, they can become sick with gastroenteritis (caused by Vibrio parahaemolyticus), cholera (caused by Vibrio cholerae) or suffer from fatal septic shock (caused by Vibrio vulnificus).

In addition to White Spot, a variety of other viral transmitted diseases can decimate shrimp farms as summarized in Table 1, Viral Diseases of Shrimp, and Table 2, Shrimp Species and Disease Symptoms (Gill 2000).

Other viral pathogens have been identified in shrimp farms around the world and have caused devastation to the industry because of their highly contagious nature. There are no known cures for viral pathogens, making prevention the only means of reducing the economic impact. (Walker 2010).

Viruses are DNA or RNA encased in protein. Viruses can be classified as naked or enveloped. The naked viruses have their DNA or RNA surrounded by a simple protein coating. Enveloped viruses are surrounded by phospholipids that they steal from the cells that they parasitize. Enveloped viruses can be rendered harmless when their viral envelope is destroyed, because the virus no longer has the recognition sites necessary to identify and attach to host cells. Additionally, enveloped viruses have protein probes projecting through their phospholipid coating. There have been several approaches to deal with viral agents, and Sagawa (U.S. Pat. No. 6,518,317) disclosed some specific organic antiviral agents.

In addition to viral diseases, shrimp colonies are susceptible to a variety of bacterial diseases. These bacterial diseases are summarized in Table 3, Bacterial Diseases of Shrimp.

Bacteria are ubiquitous microflora of seawater and are present in shrimp pond water. The accumulation of unutilized feed and shrimp fecal matter supports the multiplication of many marine bacteria species and provides substrate nutrients for the proliferation of pathogenic species. Bacterial infections of shrimp are primarily stress related such as low dissolved oxygen and high levels of mineralized nitrogen such as ammonia and nitrite. Adverse environmental conditions, sudden osmotic changes or mechanical injuries are important factors in the manifestation of bacterial disease as well. Intensive shrimp farming imposes stress on shrimp populations and makes them more susceptible to disease.

Bacterial diseases include Vibriosis, Necrotizing Hepatopancreatitis, Zoea II Syndrome, Mycobacteriosis and Rickettsial Disease and more recently Hepatopancreatic microsporidiosis (HPM) caused by the shrimp microsporidian Enterocytozoon hepatopenaei (EHP). The shrimp farming industry has been dealing and managing Vibriosis for decades. Acute Hepatopancreatin Necrosis Disease (AHPND), causative agent of Vibrio parahaemolyticus with a common name of “Early Mortality Syndrome” (EMS) almost destroyed the industry. EMS organisms represent the number one bacterial pathogen impacting today's shrimp culture industry on a worldwide basis.

Vibriosis is also known as Blackshell Disease, Septic Hepatopancreatic Necrosis, Tail Rot, Brown Gill Disease, Swollen Hindgut Syndrome, Firefly Disease and Luminous Bacterial Disease.

Necrotizing Hepatopancreatitis, NHP, also known as Texas Necrotizing Hepatopancreatitis (TNHP), Granulamatous hepatopancreatitis, Texas Pond Mortality Syndrome (TPMS), Peru Necrotizing Hepatopancreatitis (PNHP is a severe bacterial disease affecting penaeid shrimp aquaculture. NHP results in significant mortalities and devastating losses to shrimp crops.

Zoea II Syndrome has no known treatment.

Mycobacteriosis, also known as Mycobacterium Infection of Shrimp and Shrimp Tuberculosis has no proven treatment, but prolonged use of a combination of antimicrobials is thought to be effective.

Rickettsial Disease has no proven treatment.

Management protocols are being developed for Vibriosis and AHPND. However, there are always new diseases impacting the international shrimp farming industry. One example is the obligate microsporidian parasite Enterocytozoon hepatopenaei (EHP). EHP is the causative agent of hepatopancratic microsporidosis and has been reported in association with “white feces”-syndrome that causes slow growth, morbidity and or mortality and has had a severe economic impact on Asian shrimp production [Loc Tran. Second FAO Symposium on AHPND Bangkok 2016].

Natural infections have been recorded from black tiger shrimp (Penaeus monodon), Kuruma shrimp (P. japonicus), Chinese white shrimp (P. chinensis), banana prawns (P. merguiensis and P. inducus), white shrimp (P. vannamei), and other penaeids.

It is reasonable to assume that all cultured penaeids are susceptible to infection. There are about 100 penaeid shrimp species, of which a dozen of Penaeus spp. and Metapenaeus spp. have commercial value. Some shrimp types commonly cultured are: Penaeus chinensis, P. monodon, P. japonicus, P. merguinsis, P. penicillatus, Metapenaeus ensis and P. vannamei. Natural infections also occur in many species of decapods (crabs, crayfish, lobsters and shrimp) or other crustaceans, although often not lethal.

Some commercial shrimp operations discharge untreated nutrient concentrated water containing waste products directly into waterways surrounding the production facility. The nutrients discharged in shrimp effluents can negatively impact water quality in the receiving body of water. These effluents contain mineralized nutrients such as nitrogen and phosphorus that accelerate phytoplankton growth and eutrophication of the water into which it is discharged. There has been much effort devoted to alleviation of eutrophication. To reduce the impact of nutrient loading, sedimentation ponds (quiet zones) are being implemented to moderate hydraulic loading and recycled aquaculture systems (RAS) that are being developed as technology upgrades to address environmental certifications. But a concentrated industry of extensive (traditional) and intensive shrimp production ponds may be expected to contribute to environmental degradation of coastal waterways.

Excess nutrients loading from nitrogen and phosphorus and organic matter in the shrimp producing system leads to eutrophication and worsening of the culture environment. Replacing pond water volume by pumping in new water in order to maintain the water quality in the culture environment results in the discharge of concentrated organic and mineralized metabolic byproducts from feeding to the environment. Polluted seawater may become a source of diseases if pumped into other shrimp ponds. In addition, if antibiotics and disinfectants are employed by the producer for management control of shrimp diseases, these may be detected in the processed shrimp product, thus affecting product quality in the seafood market.

The conversion ratio of feed input into a traditional pond aquaculture system to shrimp biomass harvested from the system, has an average range of 1.6-2.0:1. Approximately 35% of the total feed applied to a system is excreted into the water column to accumulate as organic and mineralized residues. As much as 75% of the nitrogen and phosphorus component of shrimp feeds is discharged into natural environments in wastewater effluents from production facilities. Routine hydraulic exchange in traditional extensive shrimp pond aquaculture averages 5-10% of pond volume per day. Daily water exchange is the mandate for mediation of the negative impact from water quality degradation that results from accumulation of metabolic wastes during normal growth cycle feeding activities.

Concentrated traditional shrimp farming along a marine coastline has added to coastal pollution in many parts of the world. For example, many shrimp farms in central and eastern Thailand have failed due to pollution, according to Vaiphasa (2007). In other areas where coastal pollution has become critical, excessive amounts of chemical additives and antibiotics have been used to keep diseases from causing excessive shrimp mortality. In many cases these practices have not had success in controlling disease.

Therefore, the international aquaculture industry requires effective management protocols to control the manifestation of microbial related diseases for commercial shrimp, shellfish, and fish production, without the use of antibiotics or reducing stocking densities, or applying significant water treatment techniques, other methods, or combinations known in the art.

Management control of disease manifestation in pond systems also has the benefit of mitigating the impact of pathogens in the farm wastewater effluent that may contaminate the intake water of adjacent or regional aquaculture production facilities, a common problem in tropical coastal zones that become saturated with shrimp farm development.

TABLE 1
Exemplary Viral Diseases of Shrimp

| | | | Taxonomic | Naked or |
|---|---|---|---|---|
| | Abbreviation | Genome | classification¹ | Enveloped? |
| DNA viruses | | | | |
| Monodon | MBV | dsDNA | Baculoviridae | Enveloped |
| baculovirus | | | | |
| Baculoviral | BMNV | dsDNA | Baculoviridae | Enveloped |
| midgut gland | | | | |
| necrosis virus | | | | |
| White spot | WSSV | dsDNA | Nimaviridae, | Enveloped |
| syndrome virus | | | Whispovirus | |
| Infectious | IHHNV | ssDNA | Parvoviridae, | Naked |
| hypodermal and | | | Densovirus | |
| haematopoietic | | | | |
| necrosis virus | | | | |
| Hepatopancreatic | HPV | ssDNA | Parvoviridae, | Naked |
| parvovirus | | | Densovirus | |
| RNA viruses | | | | |
| Yellow head | YHV | (+) | Nidovirales, | Enveloped |
| virus | | ssRNA | Roniviridae, | |
| | | | Okavirus | |
| Taura syndrome | TSV | (+) | Picornavirales, | Naked |
| virus | | ssRNA | Dicistroviridae | |
| Infectious | IMNV | dsRNA | Totivirus | Naked |
| myonecrosis | | | (unclassified) | |
| virus | | | | |
| Macrobrachium | MrNV | (+) | Nodavirus | Naked |
| rosenbergii | | ssRNA | (unclassified) | |
| nodavirus (White | | | | |
| Tail Disease) | | | | |
| Laem-Singh | LSNV | (+) | Luteovirus-like | Naked |
| virus | | ssRNA | (unclassified) | |
| Mourilyan virus | MoV | (−) | Bunyavirus-like | Enveloped |
| | | ssRNA | (unclassified) | |

TABLE 2
Exemplary Shrimp Species and Disease Symptoms (Gill 2000)

| Virus | Species Affected | Disease/Symptom |
|---|---|---|
| Taura Syndrome | Penaeus | Lesions in cuticle, gills, |
| Virus (TVS) | vannamei | digestive tract and |
| | | appendages. |
| White Spot | P. vannamei, | White spots on exoskeleton, |
| Baculovirus (WSBV) | P. monodon, | appendages and other body |
| | P. japonicus, | parts. Lethargy and red |
| | P. indicies, | discolouration of |
| | P. chenensis | hepatopancreas. |
| Yellowhead Virus | P. vannamei, | Yellow discolouration of |
| (YHV) | P. monodon | carapace, gills or |
| | | hepatopancreas. Hypertrophy |
| | | of nucleii in cells of lymphoid |
| | | organ, hepatopancreas, gills |
| | | and heart. |
| Infectious Hypodermal | P. stryloristris, | Stunted growth, deformities, |
| and Haematopoietic | P. monodon | cellular inclusion bodies. |
| Necrosis Virus | (unaffected | Affects cuticular epidermis, |
| (IHHNV) | carrier) | haematocytes, haemopoietic |
| | | organs and connective tissue. |

TABLE 3
Exemplary Bacterial Diseases of Shrimp

| Disease | a.k.a. | Bacterium | Treatment |
|---|---|---|---|
| Vibriosis | Known as Blackshell | Numerous | Medicated feed at |
| | Disease, Septic | etiological agents: | 4 g/kg oxytet, |
| | Hepatopancreatic | V. harveyi, | nitrofurizolidone, |
| | Necrosis, Tail Rot, | V. vulnificus, | sarafloxathin (Sarafin); |
| | Brown Gill Disease, | V. parahaemolyticus, | Formalin at 10-25 ppm; |
| | Swollen Hindgut | V. alginolyticus, | Malachite Green |
| | Syndrome, Firefly | V. penaeicida, | at 5-10 ppb; |
| | Disease and Luminous | V. anguillarum, | EDTA at 10-50 ppm; |
| | Bacterial Disease | V. splendidus, | Furanace at 1 ppm; |
| | | V. damsela | Chloramphenical |
| | | | at 1-10 ppm; |
| | | | OTC at 1-10 ppm |
| Necrotizing | Texas Necrotizing | New genus of the | Use of oxytet at 4 |
| Hepatopancreatitis | Hepatopancreatitis | Protobacteria | g/kg (4,000 ppm), |
| | (TNHP), | (alpha) group | OTC at 0.5-2 kg |
| | Granulamatous | | per 1000 kg of feed |
| | hepatopancreatitis, | | |
| | Texas Pond Mortality | | |
| | Syndrome (TPMS), | | |
| | Peru Necrotizing | | |
| | Hepatopancreatitis | | |
| | (PNHP) | | |
| Zoea II Syndrome | | 1997: agent | No known treatment |
| | | determined to be | |
| | | intracellular | |
| | | bacteria (found by | |
| | | TEM in HP) | |
| Mycobacteriosis | Mycobacterium | Mycobacterium | No known treatment |
| | Infection of Shrimp | marinum and | but prolonged use |
| | and Shrimp | M. fortuitum | of a combination |
| | Tuberculosis | | of antimicrobials |
| | | | thought to be |
| | | | effective |
| Rickettsial Disease | Rickettsial | Rickettsia or | None reported; |
| | infection of | Rickettsia-like | tetracycline |
| | Penaeid shrimp | microorganisms | |

For the successful management of farms of aquatic animals, such as shrimp farms, the factors of predators, competitors, and pests also need to be addressed. These issues have been addressed by Harry (1978), and Section 9 of that work, which is excerpted herein.

For shrimp and shellfish, predators include insects, fish, crabs, and birds. Several of these predators will be discussed in detail for shrimp aquaculture, and they can be applied as reasonable to other shellfish and fish aquaculture systems.

For shrimp and shellfish, competitors include snails, fish, crabs, and other species of shrimp.

For shrimp and shellfish, pests include crabs, burrowing shrimp, such as Thalassina, organisms that degrade wood, mud worm egg cases, and shells.

Fish can act as a predator or as a competitor to shrimp and shellfish. The most efficient control method is to prevent them from entering the subject ponds. First, proper maintenance of the ponds is necessary to prevent fish from entering through leaks in dikes and weir boxes. Second, drying the pond bottom thoroughly before stocking with shrimp or shellfish will eliminate fish. Third, screening water as it enters the pond is an important method to control fish entry into the ponds. The screens must be sufficiently fine to prevent transport of fish eggs and larvae, as well as the adult fish. Typically, a plastic screen with a hole size of 0.2 to 0.5 millimeters is recommended, but this fine mesh is easily stopped up unless a series of screens are used. Optionally, a bag filter may be used to increase the filtration area. If a bag filter is used, then the discharge end of the bag may be connected to a floating screen box where trash will collect and can be removed by a dip net.

Crabs are one of the most destructive animals in a shrimp pond according to Harry (1978). The swimming crabs of the family Portunidae are especially destructive to shrimp—and need to be trapped. Burrowing crabs are often the source of water leakage through pond dikes. The insecticide “Sevin” historically was used for killing crabs, but it was also toxic to shrimp.

Snails compete with the shrimp for food in a pond—and according to Harry (1978) most operators feel that production is lowered with increased numbers of snails. Several commercial preparations that were historically used to kill snails include “Brestan,” “Aquatin,” and “Bayluscide.” However, these compounds are no longer in use.

Wading birds are also a predator to shrimp. If the water in the pond is kept deep enough and colored with a growth of phytoplankton, the birds cannot see the bottom and will not land. This is an effective means of control.

For other pests, such as insects, all but one of the pesticides used globally in shrimp production are banned for use in U.S. shrimp farms. Only a diluted form of formaldehyde, called formalin, is approved for U.S. shrimp farms. The FDA is capable of testing imported shrimp for residues of 360 pesticides, and shipments over the legal limit may be refused.

Organisms that degrade wood may also be a major problem for water control structures. This risk can be reduced by constructing water control structures of concrete. However, if wood construction is used, or if wood is used in concrete control structures as weir boards, then the following wood degrading organisms should be considered: Mollusca (Teredinidae or shipworms, Pholadidae or piddocks), Crustacea (Isopoda), and Fungi. The Mollusca causes damage by boring, while Fungo causes soft rot. The type of wood used can positively impact its durability, and there is a preference for those that contain high silica content (Dialium sp., Parinari sp, Licania sp, Eschweiler asp., Meirosideros sp.) or contain compounds that act as a repellent (Eusideroxylon zwaggeri, Ocotea rodiaei, Callitris glauca, Eucalyptus marginate). Preservative coatings may also be applied to wood to increase its resistance, and these treatments include Creosote. For removable parts like weir boards, more frequent application of treatments may be needed, or the use of plastics may be considered. In some cases, Polywood® or wood that has a plastic treatment may be used for weir boards.

In summary, there is also a need in the industry to be able to control the harmful impact of predators, competitors, and pests on the aquaculture of shellfish, shrimp, and fish without the need for using harmful chemicals, antibiotics or expensive treatment methods. It is thus an object of the present invention to provide a process and equipment that overcome at least some of the problems in prior art.

In most cases, microalgae co-habitat aquaculture pond systems with shrimp (U.S. Pat. No. 3,998,186), fish (U.S. Pat. No. 9,487,716), or shellfish (U.S. Pat. No. 8,753,851 B2). In these synergistic systems, the algae are typically consumed by the shrimp, fish, or shellfish as a source of nutrition. Since these species live in either fresh water, seawater, or brackish water, the salinities of the aqueous growth medium is low enough to sustain life for the shrimp, fish, or shellfish. Thus, these patents and U.S. Pat. No. 6,986,323 discuss synergies between growing algae and shrimp, fish, or shellfish at seawater salinities or below.

### SUMMARY

The processes and systems disclosed herein overcome the deficiencies in the art. In particular, in one aspect, there are disclosed processes and systems herein, wherein the growth of aquatic animals in the processes and systems generates a process stream comprising waste products and/or byproducts, which contain nutrients useful in the growth of algae (algal nutrients), such as phosphorus, nitrogen, and/or iron-containing substances. To date, known processes typically discard such process streams from the growth of aquatic animals to open water, such as lakes, rivers, seas, and oceans. However, aspects of the present invention instead utilize algal nutrients remaining in the process stream from the growth of aquatic animals for the growth of algae, thereby reducing necessary materials and costs associated with providing algal nutrients for algal growth.

In accordance with another aspect, such process streams from the growth of aquatic animals further include a plurality of pathogenic microbes, competitors, pests, and/or predators harmful to aquatic animals and other organisms. When such process streams are discarded to open water, such as rivers, estuaries, bays, and oceans, the pathogenic microbes, competitors, and/or predators may also be distributed into the receiving body of water and may cause harm to marine wildlife and pose an environmental hazard to aquatic animals.

Aspects of the invention instead direct the process stream to an algal aquaculture pond comprising an algal aquaculture medium operated at a salinity of at least about 7 wt-%. When the salinity of the process stream is increased and/or is otherwise introduced into an aqueous medium of greater salinity, the number of pathogenic microbes, competitors, pests, and/or predators in the process stream is reduced. Thereafter, if the process stream is discarded to a body of water along with algal aquaculture medium, the process stream has been treated (vs. untreated as in known processes and systems), has a reduced pathogenic microbe, competitor, pest, and/or predator count, and is more environmentally friendly.

In view of the above, in one embodiment, there is provided a process for culturing algae and/or reducing pathogenic microbes from an aqueous medium. The process comprises:
growing aquatic animals in an aquaculture pond comprising an aqueous medium comprising a salinity of 0 to 5 wt-%;
feeding a process stream generated from the growth of aquatic animals to an algal aquaculture pond; and
culturing algae in the algal aquaculture pond in an algal aquaculture medium comprising a salinity of at least 7 wt-%, wherein at least a portion of the algal aquaculture medium comprises the process stream.

Alternatively, in the above process the algal aquaculture medium comprises at least a portion of the process stream. By “at least a portion” is here meant that for example 10% by volume (vol-%) of the process stream is comprised or forms part of the algal aquaculture medium. The portion can be any suitable amounts, such as from 5 vol-% up to 99 vol-%, and the algal aquaculture medium can also consist entirely of the process stream generated from the growth of aquatic animals. The above process can also be used for treating said process stream. The process stream generated from the growth of aquatic animals is an aqueous stream, as the aquatic animals are grown in an aqueous medium.

In one embodiment, the above process further comprises harvesting the algae from the algal aquaculture pond to obtain an algal concentrate. In an embodiment, there is provided an algal concentrate or organic algal concentrate prepared from or by the process of the present invention, or obtained or obtainable by a process of the present invention.

In another embodiment, there is provided an aquaculture system for growing algae, or reducing microbes in an aqueous medium, wherein the system comprises a source of a process stream generated from the growth of aquatic animals in an aqueous medium, comprising a salinity of 0 to 5 wt-%; and an algal aquaculture pond for culturing algae in fluid communication with the source of the process stream and arranged to receive the process stream therefrom, wherein the algal aquaculture pond comprises an algal aquaculture medium comprising a salinity of at least 7 wt-%, and wherein at least a portion of the algal aquaculture medium comprises the process stream. The system further comprises means for increasing salinity of the algal aquaculture medium and/or salinity of the process stream. In one embodiment, the process stream generated from the growth of aquatic animals has a salinity of 0 to 5 wt-%.

In another embodiment, there is provided an aquaculture system for treating a process stream, growing algae, or reducing microbes in an aqueous medium, wherein the system comprises:
an aquaculture pond for growing aquatic animals, wherein the aquaculture pond for growing aquatic animals comprises an aqueous medium for growing aquatic animals having a salinity of 0 to about 5 wt-%;
an algal aquaculture pond for culturing algae in fluid communication with the aquaculture pond for growing aquatic animals, wherein the algal aquaculture pond comprises an algal aquaculture medium therein having a salinity of at least about 7 wt-%.

In another embodiment, there is provided a use of the system of the present invention for culturing algae and/or for reducing pathogenic microbes from a process stream generated from the growth of aquatic animals.

In one embodiment, the process comprises feeding the process stream to the algal aquaculture pond from one or more aquaculture ponds for the growth of the aquatic animals.

In an alternative, the process comprises feeding the process stream from more than one of such aquaculture ponds, such as from two, three, four, five, six, seven, eight, nine, ten or more aquaculture ponds.

In one embodiment, in the processes, upon feeding of the process stream to the algal aquaculture pond, the salinity of the algal aquaculture medium reduces an amount of pathogenic microbes, competitors, and/or predators harmful to aquatic animals in the process stream.

In one embodiment, the process further comprises:
feeding the algal aquaculture medium from the algal aquaculture pond to one or more further aquaculture ponds for the growth of algal aquacultures or aquatic animals; and/or
recycling at least a portion of the algal aquaculture medium from the algal aquaculture pond back to a pond for the culture of aquatic animals and combining the algal aquaculture medium with an aqueous medium that reduces a salinity of the algal aquaculture medium.

In one embodiment, the process further comprises discharging at least a portion of the algal aquaculture medium after the culturing of algae to open water, such as an ocean or a sea. In another embodiment, the process further comprises discharging at least a portion of the algal aquaculture medium after the culturing of algae to a pond used in the process for the production of solar salt, such as a crystallizer or a pre-crystallizer evaporation pond.

In one embodiment, the process further comprises adding algal nutrients to the process stream for the culturing of the algae.

In one embodiment, the process further comprises harvesting the algae from the algal aquaculture pond to obtain an algal concentrate.

In one embodiment, the process comprises preparing a product, optionally a feed product, from the obtained algal concentrate.

In one embodiment of the process, the salinity of the process stream is increased in transfer means, such as a conduit, between a growth pond within which the growth of aquatic animals takes place and the algal aquaculture pond. The salinity of the process stream can be increased by adding salt in any form, such as solid salt materials or an aqueous medium having a higher salinity than the salinity of the process stream.

The transfer means can be any suitable means for transferring the process stream from one pond to another. It may for example be a conduit, a pipe, a gutter, a canal, a channel or similar, or any combination thereof. The transfer means can have means for controlling the flow of the process stream, such as a valve, for example at its inlet (i.e. an outlet of the growth pond), at its outlet (i.e. an inlet of the algal aquaculture pond), in between these two or it may comprise several of such controlling means. If need be, the transfer means can also be equipped with a pump or some other equipment for transferring the process stream, while the transfer preferably takes place under gravity, without any external devices.

The solid salt materials can be added via a suitable inlet, such as an inlet for powder materials, while the aqueous medium having a higher salinity than the salinity of the process stream can be added via a suitable inlet, such as a valve for liquid. Optionally such a solid addition could be made by manually adding solid salt materials to an open channel, wherein water or brine are intimately contacted with the solid material to dissolve the solid salt material. Preferably, the amount of material, either solid or liquid, can be controlled with suitable controlling means. The addition can be performed at any suitable location of the transfer means, depending for example on the need for dissolving and/or mixing the added material with the process stream. This is also why it is preferred to add the salinity increasing material to the process stream before it enters the algae aquaculture ponds, i.e. to ensure that the salinity of the algae aquaculture pond remains as constant as possible over time, and also that the salinity remains as equal as possible at different points of the algal aquaculture pond. In case of liquid salinity increasing material, the addition can also be performed either close to the inlet of the algal aquaculture pond or at the inlet, as two liquid streams typically mix well under the flow. Furthermore, the means for transferring the process stream can be equipped with means for determining and/or controlling its salinity. For example, a sensor for salinity can be arranged at the inlet or in the beginning of the means for transferring, where after the required increase in salinity is determined, and the salinity increasing material is added in a corresponding amount.

In one embodiment of the processes, systems, algal concentrates, or uses, the salinity of the algal aquaculture medium and/or the process stream comprises sea salts, underground salts, salts of aquifer water, salts of a terminal lake, sodium chloride and/or any combination of ions present in sea salt.

In one embodiment, the system further comprises a source of algal nutrients arranged for introducing algal nutrients to the process stream and/or the algal aquaculture medium.

In one embodiment, the system further comprises an outlet and means for transferring, such as a conduit, arranged for discharge of at least a portion of the algal aquaculture medium e.g. to an open body of water, such as an ocean or a sea. The means for transferring can be for example any of the examples given above for the process stream. It may be necessary to discharge a portion of the algal aquaculture medium for example if there is more process stream coming into the algal aquaculture pond than what evaporates.

In one embodiment, the system further comprises means for recycling, such as a recycle conduit, from the algal aquaculture pond to the source of the process stream for recycling at least a portion of the algal aquaculture medium from the algal aquaculture pond. The means for recycling can be for example any of the examples given above for the transfer means for the process stream. The means for recycling may also need a pump, if the process stream is not transferred by gravity, in which case the algal aquaculture medium would need to be pumped to the source of the process stream. The algal aquaculture medium may also be recycled directly to the aquaculture pond.

In one embodiment of the processes, systems, algal concentrates, or uses, the source of the process stream comprises an aquaculture pond for growing aquatic animals, and the size of the aquaculture pond for growing aquatic animals is about 0.1-1000 about hectares, about 0.1-200 about hectares, about 0.1-about 100 hectares, about 0.1-about 20 hectares, about 1-about 50 hectares, about 1-about 20 hectares, about 1-about 10 hectares, or about 5-about 10 hectares, and/or wherein the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares; or the size of a pond for the growth of the aquatic animals is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares.

In one embodiment of the processes, systems, algal concentrates, or uses, the source of the process stream comprises an aquaculture pond for the growth of aquatic animals or an open aquaculture pond for the growth of aquatic animals; or a pond for the growth of the aquatic animals is an open pond.

In one embodiment, the system is configured to carry out the processes of the present invention.

In one embodiment of the processes, systems, algal concentrates, or uses, the algal aquaculture pond is an open pond.

In one embodiment of the processes, systems, algal concentrates, or uses, the algal aquaculture medium comprises a salinity of or the salinity of the algal aquaculture medium is at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%, or at least about saturation.

In one embodiment of the processes and systems, the difference between the salinities of the aqueous medium of the aquaculture pond (used for culturing aquatic animals) and the algal aquaculture medium of the algal aquaculture pond (used for culturing algae) can be e.g. at least 2 wt-%, at least 3 wt-%, at least 4 wt-%, at least 5 wt-%, at least 6 wt-%, at least 7 wt-%, at least 8 wt-%, at least 9 wt-%, at least 10 wt-%, at least 11 wt-%, at least 12 wt-%, at least 13 wt-%, at least 14 wt-%, at least 15 wt-%, at least 16 wt-%, at least 17 wt-%, at least 18 wt-%, at least 19 wt-%, at least 20 wt-%, at least 21 wt-%, at least 22 wt-%, at least 23 wt-%, at least 24 wt-%, or at least 25 wt-%. The difference between the salinities can be for example from at least 2 wt-% up to 25 wt-%, or from at least 2, 3, 4, 5, 6, 7, 8, 9, 10, 11, 12, 13, 14, 15, 16, 17, 18, 19, 20, 21 or 22 wt-% up to 4, 5, 6, 7, 8, 9, 10, 11, 12, 13, 14, 15, 16, 17, 18, 19, 20, 21, 22, 23, 24, or at least 25 wt-%.

In one embodiment of the processes, systems, algal concentrates, or uses, the salinity of the algal aqueous medium of the algal aquaculture pond is from about 15 wt-% or from about 20 wt-% to saturation.

In one embodiment of the processes, systems, algal concentrates, or uses, the process stream comprises a salinity of from 0 to about 5 wt-%, such as less than about 0.5 wt-%, less than about 1 wt-%, less than about 1.5 wt-%, less than about 2 wt-%, less than about 2.5 wt-%, less than about 3 wt-%, less than about 3.5 wt-%, less than about 4 wt-%, or less than about 4.5 wt-%, or e.g. 1-5 wt-%, 1-4 wt-%, 1-3 wt-%, or 1-2 wt-%.

In one embodiment of the processes, systems, algal concentrates, or uses, the process stream comprises a waste stream or a recycle stream generated from the growth of aquatic animals.

In one embodiment of the processes, systems, algal concentrates, or uses, the aquatic animals are selected from the group comprising or consisting of crustaceans, shrimps, fishes, molluscs, shellfishes, and any combination thereof, or the aquatic animals are selected from the group comprising or consisting of Penaeid family shrimps, Penaeus chinensis, P. monodon, P. japonicus, P. merguinsis, P. penicillatus, Metapenaeus ensis, P. vannamei and Litopenaeus vannamei.

In one embodiment of the processes, systems, algal concentrates, or uses, the process stream is from ponds growing Penaeid family shrimps, including Penaeus chinensis, P. monodon, P. japonicus, P. merguinsis, P. penicillatus, Metapenaeus ensis, P. vannamei and Litopenaeus vannamei.

In one embodiment of the processes, systems, algal concentrates, or uses, the process stream comprises:
one or more of the viruses selected from the group consisting of Monodon baculovirus, Baculoviral midgut gland necrosis virus, White spot syndrome virus, Infectious hypodermal and haematopoietic necrosis virus, Hepatopancreatic parvovirus, Yellow head virus, Taura syndrome virus, Infectious myonecrosis virus, Macrobrachium rosenbergii nodavirus (White Tail Disease), Laem-Singh virus, and Mourilyan virus; and/or
one or more bacteria contributing to one or more selected from the group consisting of Blackshell Disease, Septic Hepatopancreatic Necrosis, Tail Rot, Brown Gill Disease, Swollen Hindgut Syndrome, Firefly Disease, Luminous Bacterial Disease, Texas Necrotizing Hepatopancreatitis (TNHP), Granulamatous hepatopancreatitis, Texas Pond Mortality Syndrome (TPMS), Peru Necrotizing Hepatopancreatitis (PNHP), Mycobacterium Infection, Shrimp Tuberculosis, and Rickettsial infection.

In one embodiment of the processes, systems, algal concentrates, or uses, the process stream comprises algal nutrients (e.g., nutrients added to the stream or nutrients not added to the stream) therein for the culturing of the algae.

In one embodiment of the processes, the process stream is blended with other algal nutrients.

In one embodiment of the processes, systems, algal concentrates, or uses, the algae or microalgae is selected from the group comprising or consisting of the prokaryotes Aphanothece halophytica (aka Coccochloris elabens, Cyanothece, Halothece), Microcoleus chthonoplastes; M. lyngbyaceus, Spirulina major; S. platensis, Nodularia spumigena, Dactylococcopsis salina, Synechocystis DUN52, and PCC 6803, Synechococcus PCC 7418, Phormidium spp. (e.g. P. ambiguum, P. tenue), Oscillatoria spp. (e.g. O. neglecta, O. limnetica, O. salina), Lyngbya spp. (e.g. L. majuscula, L. aestuarii), Halospirulina tapeticola, Microcystis spp., Nostoc spp., Aphanocapsa spp., and the Eukaryotes Dunaliella spp. (D. salina, D. viridis, D. parva, etc.), Dangeardinella saltitrix, Chlorella vulgaris, Navicula spp., Amphora spp. and combination thereof.

In one embodiment of the processes, systems, algal concentrates, or uses, the algae or microalgae is selected from the group of, or comprises:
one or more microalgal species selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., and Amphora spp.; or
genetically-engineered varieties of any of the above; or
any combinations thereof.

In a specific embodiment, the algae or microalgae is selected from the group comprising or consisting of Dunaliella sp., Dunaliella bardawil, Dunaliella salina, Dunaliella tertiolecta, Dunaliella parva and Dunaliella viridis, and any combination thereof.

In one embodiment of the processes, systems, algal concentrates, or uses, the algae or algal biomass of the algal aquaculture obtained with the processes of the present invention contains minimal or very low levels of pathogenic microbes that are harmful to aquatic animals, such as shrimp and fish.

In one embodiment of the processes, systems, algal concentrates, or uses, the obtained algal biomass or concentrate has a variable content of 3- and 6-omega fatty acids and their corresponding esters in conjunction with proteins and carbohydrates, and optionally carotenoids suitable for shrimp, fish and other aquatic or marine organisms.

In one embodiment of the processes, systems, algal concentrates, or uses, the salinity of the algal aquaculture medium of the algal aquaculture pond(s) comprises sea salts, underground salts, salts of aquifer water, salts of a terminal lake, sodium chloride, and/or any combination of ions commonly present in sea salt.

In one embodiment of the processes, the residence time of the process stream in the algal aquaculture pond(s) is at least about one day or at least about two days, e.g. from about one day to about four weeks, from about one day to about two weeks, from about two days to about four weeks, or from about two days to about two weeks.

### BRIEF DESCRIPTION OF THE DRAWINGS

Some of the features and advantages of the disclosure have been stated. Other advantages will become apparent as the description of the disclosure proceeds, taken in conjunction with the accompanying drawings, in which:

FIG. 1 is a schematic diagram showing a system or process for culturing algae and/or reducing pathogenic microbes from an aqueous medium in accordance with one aspect.

FIG. 2 is a schematic diagram showing a system or process that provides added nutrients and salts to the process stream and/or algal aquaculture medium in accordance with another aspect.

FIG. 3 is a schematic diagram showing a system or process that recycles algal aquaculture medium from the algal aquaculture pond(s) to the aquaculture pond(s) for growing aquatic animals in accordance with another aspect.

FIG. 4 reveals results of Examples 2-4 after treating samples from aquaculture ponds with aqueous media having different salinities.

### DETAILED DESCRIPTION

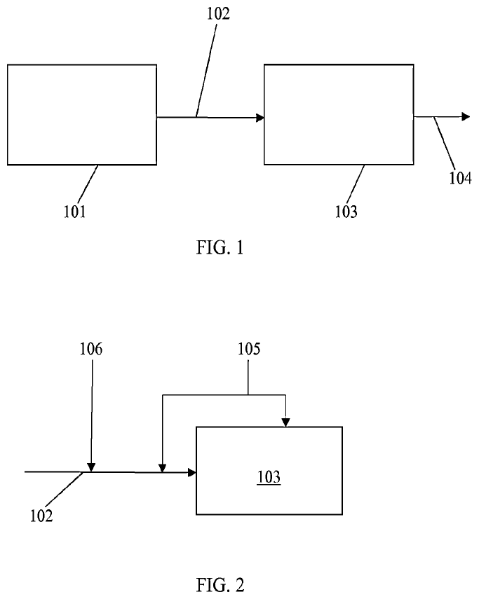

Turning now to FIG. 1, there is shown an embodiment of a process and system for culturing algae and/or reducing pathogenic microbes from an aqueous medium. In particular, there is shown a process stream 102 generated from the growth of aquatic animals being fed to one or more algal aquaculture ponds 103 (hereinafter “algal aquaculture pond(s)”) which are optionally located within an algal aquaculture facility. The processes and systems described herein may comprise one, two, three, or a further multiplicity of algal aquaculture ponds.

In an embodiment, the process stream 102 is fed to the algal aquaculture pond(s) 103 from one or more outlets of one or more aquaculture ponds 101 in which fish, shrimp, or other aquatic species are grown (hereinafter “aquaculture pond(s) for growing aquatic animals 101” or “aquaculture pond(s) 101”). Such aquaculture pond(s) 101 include a suitable volume of aqueous medium for the growth of the fish, shrimp, or other aquatic animals or species therein. In an embodiment, the aqueous medium of the aquaculture pond(s) 101 have a salinity of from 0 to about 5 wt-%.

As used herein, wt-% refers a dry mass of a component in a solution in grams divided by 100 grams of the solution. In addition, unless otherwise stated herein or clear from the context, any percentages referred to herein are understood to refer to wt-%.

As used herein, the term “about” refers to a value that is #1% of the stated value. In addition, it is understood that reference to a range of a first value to a second value includes the range of the stated values, e.g., a range of about 1 to about 5 also includes the more precise range of 1 to 5. Further, it is understood that the ranges disclosed herein include any selected subrange within the stated range, e.g., a subrange of about 50 to about 60 is contemplated in a disclosed range of about 1 to about 100.

In one embodiment, gravity is utilized for feeding the process stream 102 to the algal aquaculture pond(s) 103 from the aquaculture pond(s) for growing aquatic animals 101.

In an embodiment, the process stream 102 comprises a waste stream from the aquaculture pond(s) 101. In an embodiment, the process stream 102 comprises at least a portion of the aqueous medium in or from the aquaculture pond(s) 101 that optionally remains after the harvesting or removal of grown fish, shrimp, or other aquatic species therefrom. In another embodiment, at least a portion of the process stream 102 comprises a recycle stream from the growth of aquatic animals-meaning that the recycle stream has been used previously at least once in the growth of aquatic animals and has optionally had aquatic animals harvested therefrom.

Referring again to FIG. 1, the process stream 102 is fed to the algal aquaculture pond(s) 103 which optionally includes an amount of algal aquaculture medium therein within which algae are grown. The algal aquaculture pond(s) 103 are operated at hypersaline conditions, i.e. in the processes or systems of the present invention the salinity of the algal aquaculture medium of the algal aquaculture pond(s) 103 is at least about 7 wt-%, at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 w-%, at least about 20 wt-%, at least about 21 wt-%, at least about 22 wt-%, at least about 23 wt-%, at least about 24 wt-%, at least about 25 wt-% (e.g. up to saturation) for the growth of algae therein. The above values represent the salinity of the algal aquaculture medium in the algal aquaculture pond(s) 103 comprising at least an amount of the process stream 102 added to the algal aquaculture pond(s) 103.

In certain embodiments, the algal aquaculture medium is saturated with salt. In particular embodiments, the salinity of the algal aquaculture medium is from about 7 wt-% to saturation, from about 8 wt-% to saturation, from about 9 wt-% to saturation, from about 10 wt-% to saturation, from about 20 wt-% to saturation, about 7 w-% to about 20 wt-%, about 8 wt-% to about 20 wt-%, about 9 wt-% to about 20 wt-%, about 10 wt-% to about 20 wt-%, about 10 wt-% to about 15 wt-%, about 12 wt-% to about 25 wt-%, about 15 wt-% to about 25 wt-% or about 20 wt-% to about 25 wt-%. The salinity of the process stream 102 and/or the algal aquaculture medium in the algal aquaculture pond(s) 103 may comprise any suitable salts for providing the desired salinity. In an embodiment, the salinity comprises sea salts, underground salts, salts of aquifer water, salts of a terminal lake, sodium chloride, and/or any combination of ions present in sea salt. A mixture of sources for the salinity can also be used, in case for example more than one type is readily available.

In an embodiment, the process stream 102 has a salinity of from 0 to about 5 wt-% while the algal aquaculture pond(s) 103 have a salinity of the at least about 7 wt-% when, i.e. after, the process stream is fed to the algal aquaculture pond(s) 103. When the process stream 102 comprises a number of pathogenic microbes, competitors, and/or predators harmful to aquatic animals therein, upon an increase in the salinity of process stream 102 resulting from addition to the algal aquaculture pond(s) 103 or otherwise, the increase in salinity of the process stream 102 and/or contact with a medium of greater salinity is effective to reduce an amount of the pathogenic microbes, competitors, and/or predators harmful to aquatic animals originating from the process stream 102. The mechanisms by which this effect occurs or may occur are explained in further detail below.

As mentioned previously, aspects of the processes and systems also advantageously utilize nutrients from the process stream 102 for the growth of algae in the algal aquaculture pond(s) 103, instead of immediately discarding the process stream 102 to the environment as in known processes and systems. In certain embodiments, as shown in FIG. 2, additional nutrients 105 may be added to the process stream 102 or to the algal aquaculture pond(s) 103 to supplement the nutrients found in the process stream 102. In an embodiment, the additional nutrients 105 are added to the process stream 102 upstream of the algal aquaculture pond(s) 103. In another embodiment, additional nutrients 105 are added to the algal aquaculture pond(s) 103 following or simultaneous with the addition of the process stream 102. The additional nutrients may include nitrogen, phosphorus, iron, or any other suitable species for promoting the growth of algae, e.g., sulfur and manganese, copper, zinc, molybdenum and boron. Suitable nitrogen sources include, but are not limited to ammonia, urea, nitrates, or combinations thereof. Suitable phosphorus sources include, but are not limited to phosphoric acid, diammonium phosphate, phosphates, and other sources of phosphorus. Suitable iron sources include e.g. EDTA chelated iron, and other soluble and insoluble forms of iron. Many of the abovementioned micronutrients are contained in seawater and other sources of water.

In certain embodiments, as is also shown in FIG. 2, the salinity of the process stream 102 may also or instead be increased (shown as arrow 106) prior to delivery of the process stream 102 to the algal aquaculture ponds 103. The increase in salinity may be done by the addition of salt in any form, such as the addition of solid salt materials or the addition of an aqueous medium having a higher salinity than that of the process stream 102, or both (either at different locations or at the same location). This step adjusts the salinity of the process stream 102 fed to the algal aquaculture pond(s) 103 closer to the salinity of the algal aquaculture pond(s) 103, and may also serve to reduce a number of pathogenic microbes, competitors, and/or predators harmful to aquatic animals in the process stream 102.

In certain embodiments, as shown, both algal nutrients and salts may be added to the process stream 102 before feeding of the process stream 102 to the algal aquaculture pond(s) 103. In an embodiment, the addition of either or both of the algal nutrients and salts to the process stream 102 is provided from suitable sources thereof in a conduit in fluid connection between the aquaculture pond(s) for the growth of aquatic animals 101 and the algal aquaculture pond(s) 103. In one embodiment, the salinity of the process stream 102 is increased in a transfer means, such as a conduit, between the aquaculture pond(s) 101 and the algal aquaculture pond(s) 103. The transfer means may be of any suitable structure, size, and shape for the delivery of the process stream 102 with the added materials (nutrients and/or salts), also as explained above. In certain embodiments, the conduit may be open to the atmosphere, such as a channel extending from the aquaculture pond(s) for the growth of aquatic animals 101 and the algal aquaculture pond(s) 103. In other embodiments, such a conduit may be provided for the feeding of the process stream 102 to the algal aquaculture pond(s) 103 without the addition of salts or additional nutrients.

Following growth of the algae in the algal aquaculture pond(s) 103, the algal aquaculture medium may be discharged from the algal aquaculture pond(s) 103 (shown by arrow 104 in FIG. 1). In certain embodiments, grown algae in the algal aquaculture pond(s) 103 is harvested from the algal aquaculture medium before being discharged from the algal aquaculture pond(s) 103. In other embodiments, grown algae is harvested from the algal aquaculture medium after being discharged from the algal aquaculture pond(s), such as in one or more harvesters located downstream of the algal aquaculture pond(s) 103.

In one embodiment, the process may further include feeding the algal aquaculture medium from the algal aquaculture pond(s) 103 to one or more further aquaculture ponds for the growth of algae or aquatic animals therein. In the case of further aquaculture ponds for the growth of algae, the algal aquaculture medium may be combined with any further streams necessary for the growth of the algae or to provide the desired conditions for additional algae growth. In the case of further aquaculture ponds for the growth of aquatic animals, the algal aquaculture medium discharged from the algal aquaculture pond(s) 103 may also be combined with any suitable aqueous stream to reduce a salinity of the algal aquaculture medium to a salinity, e.g., 0 to about 5 wt-%, suitable for the growth of aquatic animals.

In an embodiment, the process may further comprise harvesting the algae from the algal aquaculture pond(s) 103 or further aquaculture ponds to produce an algal concentrate. In an embodiment, the algal concentrate comprises or is an organic algal concentrate. By “organic algal concentrate,” it is meant that the algae has been grown in an algal aquaculture medium that comprises nutrients generated from a biological process as described herein vs. a chemical process which generates the nitrogen and phosphorus species for algal growth from natural gas, and thereafter at least part of the algal aquaculture medium has been removed to obtain an organic algal concentrate. The algal aquaculture medium can be removed or separated from the algae e.g., by a harvester. In this way, aspects of the present invention may reduce greenhouse gas generation from chemical processes that would otherwise be used to provide algal nutrients necessary for the growth of algae.

In one embodiment, the system further comprises an algal harvester connected to, following, or downstream of the algal aquaculture pond(s) 103.

In another embodiment, the process may further include the step of recycling at least a portion of the algal aquaculture medium from the algal aquaculture pond(s) 103 back to the aquaculture pond(s) 101 (i.e. the source of the process stream 102) for the growth of further aquatic animals in the aquaculture pond(s) 101 as shown in FIG. 3. In this embodiment, the process may further include the step of combining the algal aquaculture medium recycled (shown as 107) from the algal aquaculture pond(s) 103 with additional aqueous medium 108 that reduces a salinity of the algal aquaculture medium.

In another embodiment, the process may further include the step of recycling at least a portion of the algal concentrate from the harvester back to the aquaculture ponds for the growth of aquatic animals. In one embodiment, the system comprises means for recycling, such as a recycle conduit, arranged to recycle the algal concentrate from the algal harvester to the aquaculture pond for aquatic animals. The means for recycling is as described above.

In another embodiment, following growing of an amount of algae in the algal aquaculture medium, the process further comprises discharging at least a portion of the algal aquaculture medium to open water, such as an ocean or a sea. As discussed previously herein, the discharged algal aquaculture medium comprises at least a portion of the process stream 102, which has been treated to reduce pathogenic microbes, competitors, and/or predators harmful to aquatic animals therein.

In certain embodiments, the process may be operated as a continuous process in that the process stream 102 may be continuously fed into the algal aquaculture pond(s) 103 as algal aquaculture medium is discharged from the algal aquaculture pond(s) 103. In other embodiments, the process may be operated as a semi-continuous process. In one embodiment, the systems may be for a continuous or semi-continuous process.

In the process or system, the aquaculture pond(s) for growing aquatic animals 101 may be from about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or the size of the algal aquaculture pond(s) 103 may be about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares.

The algal aquaculture pond(s) 103 may comprise any suitable pond for growing algae, including but not limited to fermentation units, enclosed photobioreactors, open-pond bioreactors, and combinations thereof as are known in the art. Many types of algal ponds have been proposed in the art, and the subject is currently an area of intense research. Suitable algal ponds generally fall into three categories: fermentation units, enclosed photobioreactors, and open-pond bioreactors. Fermentation units are commonly considered for the growth of genetically modified algae that are heterotrophic. The fermentation unit is typically constructed of steel and involves sophisticated process control. This type of algal bioreactor is appropriate for high-value products, such as docosahexaenoic acid (DHA), produced by DSM. However, this type of bioreactor is extremely expensive for the production of lower-value chemicals, such as biofuels because of the enormous capital cost of the fermentation equipment. Furthermore, the fermentation process will typically require a source of sugar, which adds costs, especially when lower-value products are being produced.

The algal aquaculture pond(s) 103 or the aquaculture pond(s) for growing aquatic animals 101 may be either lined or unlined. Unlined ponds comprise earthen borders and pond floors. Suitable liner material is either plastic or clay. Plastic pond liners are typically formed from polyethylene, polypropylene, or polyvinyl chloride. Different types of these basic polymers can be used, for example linear low-density polyethylene liners are occasionally used for algae cultivation at large scale. These liners may also comprise additives, such as carbon black to provide resistance to ultraviolet radiation. These liners may also comprise Nylon™ or other fibers to provide additional structural integrity. Raven Industries (South Dakota) provides a full line of suitable liners that comprise one or more layers of materials. Suitable clay liners include bentonite clay. However, when ponds are flooded, components in the water can often form a barrier that seals the pond. It may also be desirable to include liners in just a portion of the pond where it is specifically needed. For example, to protect earthen borders where the hydraulic flow rate may be elevated. Typically, weir boxes are used for hydraulic flow control in and out of the pond. Weir boxes may be constructed from concrete, wood, high density polyethylene, other materials, or combinations thereof. They may also be fitted with slots to hold screens or barriers to impede flow, or they may have submerged weirs utilizing either holes or slots for flow control openings. Sluice gate valves, e.g., inverted Cippoletti, may also be used for flow control into a pond. The bottom of the aquaculture ponds typically have a slight slope towards the exit of the pond, but that is not essential for the instant invention. In an embodiment, the slope is about 0.5% or more, such as from about 2-3%. Borders that separate one pond from the other are typically of earthen construction, but may comprise rock, concrete, blocks, and other materials to stop the flow of water. Typically, the borders are constructed in such a way that a vehicle may be driven on top of the border. These vehicles include trucks, pick-up trucks, all-terrain-vehicles (ATVs), bicycles, and automobiles.

The aquaculture pond(s) 103 for growing aquatic animals 101 may be operated in either extensive or intensive mode. The extensive mode of operating ponds is a traditional low stocking density operating mode. For example, shrimp aquaculture ponds that are operated in the extensive mode are constructed of earthen borders that are typically unlined. Seawater is typically used to flush salt from the pond so that the salinity in the shrimp pond remains closer to that of seawater. However, this flushing also results in the discharge of some portion of the shrimp pond bottom sediment debris into the environment. The water level in extensive ponds is typically less than about one meter. In the intensive mode of operation, the pond(s) 101 can be lined with a plastic liner, and air may be added in order to mix the ponds and improve oxygen transport. The pond depth in intensive aquaculture typically averages one meter, rarely reaching 1.5 meters in depth. Stocking of shrimp can be about ten to twenty times higher in ponds that are operated in the intensive mode than ponds operated in the extensive mode.

Shrimp pond sediment debris comprises fecal solids, waste feed particles and mineralized residues. Fecal material and byproducts from protein metabolism in the shrimp's gastrointestinal tract comprises non-digested organic solids, nitrogen, phosphorus, and other micronutrients that are beneficial to algal growth. Shrimp feeds and unconsumed feed components typically comprise protein, oils, vitamins, minerals, and other materials. Some of these may be consumed directly by the shrimp, while others are consumed by predators, competitors, and pests that co-exist in the shrimp aquaculture. The shrimp pond sediment debris is composed of fecal solids, wasted feed particles and mineralized residues. Fecal material and byproducts from protein metabolism in the shrimp's gastrointestinal tract is comprised of non-digested organic solids, nitrogen, phosphorus, and other micronutrients and vitamins that are beneficial to algal growth. Shrimp feeds and unconsumed feed components are comprised of proteins, lipids, vitamins, minerals, and other materials. Wasted nutrients generated by feeding activities become concentrated in the wastewater discharge from aquaculture production facilities.

A representative chemical profile of nutrient concentrated wastewater effluent discharged from high-density shrimp aquaculture systems has been analyzed and demonstrated to be composed of the following parameters: total nitrogen+/−260 mg/liter, ammonia nitrogen+/−46 mg/liter, nitrite nitrogen+/−0.06 mg/liter, nitrate nitrogen+/−126 mg/liter, total phosphorus+/−173 mg/liter, phosphate phosphorus+/−40 mg/liter, biological oxygen demand+/−1350 mg/liter, chemical oxygen demand+/−3740 mg/liter, and total volatile solids >7,000 mg/liter.

The aquaculture pond(s) 101 for growing aquatic animals may also be smaller in volume than those previously described and may be located indoors and the shrimp or fish may be grown in specific vessels fabricated of steel, plastic, concrete, glass, plexiglass, polyethylene, fiberglass, or other materials typically used for either shrimp or fish aquaculture.

The process stream 102 from the aquatic animals, shrimp or fish aquaculture system may flow at different rates throughout the day, week, and month-depending on the growing conditions being used. Thus, it is preferable to be able to monitor the nutrients available in the process stream 102 on a continuous basis. It is preferable to monitor the nitrogen and phosphorus content of the process stream 102 so that the amount of these nutrients can be matched with the target algal productivity in the algal aquaculture pond(s).

In an embodiment of the processes or systems, the aquatic animals in the aquaculture pond(s) for growing aquatic animals 101 are selected from the group consisting of crustaceans, shrimps, fishes, molluscs, shellfishes, and any combination thereof. In particular embodiments, the aquatic animals are selected from the group consisting of Penaeid family shrimps, Penaeus chinensis, P. monodon, P. japonicus, P. merguinsis, P. penicillatus, Metapenaeus ensis, P. vannamei and Litopenaeus vannamei.

Water will evaporate from the algal aquaculture pond(s) 103, and in one embodiment this amount of water will need to be added back to the algal aquaculture pond(s) 103 in order to maintain constant salinity. Thus, it can be important to monitor the amount of water in the process stream 102. Water in this stream may be used to offset at least some of the water that evaporates from the algal aquaculture pond(s) 103.

Salt can be purged from the algal aquaculture pond(s) 103 so that it does not accumulate and change salinity in the pond(s). Thus, it can be important to monitor the amount of salt in the process stream 102 so that the proper salt purge rate may be maintained.

The salt content, by weight, of the hypersaline medium used for the growth medium in the algal aquaculture pond(s) 103 can be as much as 7.4 times saltier than the large oceans, which usually have a salinity level of 3.2 to 3.5%. However, at these high salinities, the algal growth rates are reduced, and that is typically undesired. More preferable are hypersalinity contents about 7 wt-% or more, such as about 10 wt-% or more, and this salinity level is needed to provide sufficient osmotic shock to reduce the level of pathogenic microbes, predators, and/or competitors in the process stream 102. However, at this level of salinity, a significant number of algal predators may survive that negatively impact algal growth. Thus, even more preferable are hypersalinity contents from about 12 wt-% to salt saturation or from about 15 wt-% to salt saturation so that the salinity level is high enough to exclude some competitive algal species, if that is a desired goal.

Aqueous media of hypersalinity are effective in destroying, via changing osmotic pressure, pathogenic microbes, including but not limited to bacteria that are acclimated to seawater salinity (3.5% salts by weight). When such pathogenic microbes are in a hypersaline solution, the concentration of water in the hypersaline solution is less than that inside the microbial cell. Because of the osmotic pressure difference, water tends to leave the cell. This causes the cell to dehydrate, and it eventually kills the microbe.

Suitable bacteria for treatment by the processes or systems include, but are not limited to, bacteria contributing to Blackshell Disease, Septic Hepatopancreatic Necrosis, Tail Rot, Brown Gill Disease, Swollen Hindgut Syndrome, Firefly Disease, Luminous Bacterial Disease, Texas Necrotizing Hepatopancreatitis (TNHP), Granulamatous hepatopancreatitis, Texas Pond Mortality Syndrome (TPMS), Peru Necrotizing Hepatopancreatitis (PNHP), Mycobacterium Infection, Shrimp Tuberculosis, Rickettsial infection, and combinations thereof.

While not wishing to be bound by theory, it is believed that bacteria are inactivated due to the extreme changes in osmotic pressure in going from a salt concentration about 3.5% to the much higher salinity of the algal aquaculture medium. Likewise, in concentrated solution of sea salts, the present inventors believe the protein coatings of viruses that protect their RNA or DNA are denatured and the viruses rendered inactive by the hypersaline algal aquaculture medium of the algal aquaculture pond.

Proteins are complex organic macromolecules that contain carbon, hydrogen, oxygen, nitrogen, and usually sulfur and are composed of one or more chains of amino acids. Proteins are fundamental components of all living cells and include many substances, such as enzymes, hormones, and antibodies that are necessary for the proper functioning of an organism.

Viruses are DNA or RNA encased in protein. Viruses can be classified as naked or enveloped. Naked viruses have their DNA or RNA surrounded by a simple protein coating. Their exposed protein coating is easily accessible to a chaotropic agent. Enveloped viruses are surrounded by phospholipids that they steal from the cells they parasitize. Enveloped viruses can be rendered harmless when their viral envelope is destroyed, because the virus no longer has the recognition sites necessary to identify and attach to host cells. Enveloped viruses have protein probes projecting through their phospholipid coating.

Denaturation occurs when the bonding interactions responsible for the secondary structure (hydrogen bonds to amides) and tertiary structure are disrupted. In tertiary structure there are four types of bonding interactions between “side chains” including: hydrogen bonding, salt bridges, disulfide bonds, and non-polar hydrophobic interactions that may be disrupted. Therefore, a variety of reagents and conditions can cause denaturation by application of some external stress or compound, such as a strong acid or base, a concentrated inorganic salt, an organic solvent (e.g., alcohol or chloroform), or heat. If proteins in a living cell are denatured, this results in disruption of cell activity and possibly cell death.

A chaotropic agent is a substance which disrupts the three-dimensional structure in macromolecule, such as protein, DNA (Deoxyribonucleic acid) or RNA (Ribonucleic acid), and denatures them. Chaotropic agents interfere with stabilizing intramolecular interactions mediated by non-covalent forces, such as hydrogen bonds and van der Waals forces.

Known chaotropic reagents include, but are not limited to Urea at 6-8 molarity, Thiourea at a molarity of 2, Guanidiniium chloride at 6 molarity and Lithium Perchlorate of 4.5 molarity. These reagents are expensive and have a variety of other undesirable characteristics that render them unsuitable for discharge into the environment.

The present inventors have discovered that a hypersaline media (i.e., algal aquaculture medium), concentrated (e.g. at least about 7% by weight, such as at least about 10% by weight) solutions of sea salts, are chaotropic reagents. Hypersaline media are effective both in killing bacteria via osmotic pressure changes and in deactivating viruses by denaturing the protein coating surrounding the DNA and/or RNA of the viruses. The use of hypersaline media as a chaotropic reagent avoids the costs and undesirable characteristics of prior art chaotropic reagents that render them unsuitable for discharge into the environment. And unlike prior art chaotropic reagents, the hypersaline media is not consumed, nor is its controlled discharge into the environment problematic.

Viral diseases of cultured shrimp that may be deactivated (killed) by the processes and systems include, but are not limited to, the DNA viruses more of the viruses Monodon baculovirus, Baculoviral midgut gland necrosis virus, White spot syndrome virus, Infectious hypodermal and haematopoietic necrosis virus, Hepatopancreatic parvovirus, Yellow head virus, Taura syndrome virus, Infectious myonecrosis virus, Macrobrachium rosenbergii nodavirus (White Tail Disease), Laem-Singh virus, Mourilyan virus.

RNA viruses of cultured shrimp that may be deactivated (killed) by the processes or systems include, but are not limited to, Yellow head virus, Taura syndrome virus, Macrobrachium rosenbergil nodavirus (White Tail Disease), Laem-Singh virus, Mourilyan virus and White spot syndrome virus.

Bacteria that may be deactivated (killed) by the processes or systems include, but are not limited to: Vibriosis, Necrotizing Hepatopancreatitis, Zoea II Syndrome, Mycobacteriosis and Rickettsial Disease.

Vibriosis is also known as Blackshell Disease, Septic Hepatopancreatic Necrosis, Tail Rot, Brown Gill Disease, Swollen Hindgut Syndrome, Firefly Disease and Luminous Bacterial Disease.

Necrotizing Hepatopancreatitis, NHP, also known as Texas Necrotizing Hepatopancreatitis (TNHP), Granulamatous hepatopancreatitis, Texas Pond Mortality Syndrome (TPMS), Peru Necrotizing Hepatopancreatitis (PNHP) is a severe bacterial disease affecting penaeid shrimp aquaculture. NHP results in significant mortalities and devastating losses to shrimp crops. Elevated salinity and temperature above that in typical shrimp aquaculture appear to be stress factors for the shrimp and are associated with NHP outbreaks. The magnitude of the elevated salinities in shrimp aquaculture are just slightly elevated over seawater, with values of about 4 to 5 wt-%.

Zoea II Syndrome has no known treatment.

Mycobacteriosis, also known as Mycobacterium Infection of Shrimp and Shrimp Tuberculosis has no other proven treatment but prolonged use of a combination of antimicrobials is thought to be effective.

Rickettsial Disease has no proven treatment.

Feeding aquaculture pond wastes to one or more algal aquaculture ponds reduces environmental pollution, makes effective use of available nutrients, and concurrently deals with the problem of bacteria and viruses in the waste that may be transmitted to other marine aquaculture facilities. In one embodiment, the processes, systems or uses are able to reduce the number of pathogenic microbes harmful to aquatic animals present in the algal aquaculture medium or the process stream generated from the growth of aquatic animals by at least about 10%, at least about 20%, at least about 30%, at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or even more.

The aquaculture waste may be fed directly to one or more algal aquaculture ponds for treatment or it may be blended with incoming or recycle streams optionally to provide any additional nutrients needed.

The concentration of sea salts in the algal aquaculture medium of the algal aquaculture pond(s) 103 can range from about 7 wt-% to saturation, such as from about 10 wt-% to saturation. Sea salts include sodium chloride or any combination of inorganic ions commonly present in salt from the sea. However, for the growth of specific algae, a specific salinity target may be preferred. For example, if Dunaliella salina is the preferred algal species, then in one embodiment the salinity of its growth medium can be above about 16 wt-% salinity in order to effectively exclude predators and/or competitors. Examples of such predators are brine shrimp and hetroamoeba. Further examples of such competitors are Dunaliella viridis and other Dunaliella minutia. Furthermore, it is desirable to maintain a salt concentration as low as possible in order to increase the growth rate of Dunaliella salina. Thus, an optimum exists for the salinity of the algal growth system. In addition to these constraints on salinity, the chosen salinity significantly impacts the product composition that is generated by Dunaliella salina. In a specific embodiment, as the salt concentration of the algal aquaculture medium increases from about 18 wt-% to about 25 wt-%, the Lutein/Beta-Carotene ratio can change by a factor of two or more. Thus, selecting the proper salinity of the algal aquaculture pond(s) 103 to receive the process stream 102, such as a waste or recycle stream is important.

Residence time in the high salinity algal aquaculture pond(s) 103 can be e.g. at least about 12 hours, at least about 24 hours, at least about 48 hours, from about one day (i.e. about 24 hours) to about two weeks or more, from about one day (i.e. about 24 hours) to about four weeks or more, or from about two days (i.e. about 48 hours) to about four weeks or more, in part depending on the algae growth rate and harvesting demands.

Competitors from shrimp ponds that can be reduced in concentration or eliminated entirely by the hypersaline media include, but are not limited to snails, burrowing shrimp (Thalassina), fish, Mud worm egg cases and crabs.

In one embodiment of the processes or systems, the algae is marine algae or microalgae and can be selected from the group comprising or consisting of Prokaryotes Aphanothece halophytica (aka Coccochloris elabens, Cyanothece, Halothece), Microcoleus chthonoplastes; M. lyngbyaceus, Spirulina major; S. platensis, Nodularia spumigena, Dactylococcopsis salina, Synechocystis DUN52, and PCC 6803, Synechococcus PCC 7418, Phormidium spp. (e.g. P. ambiguum, P. tenue), Oscillatoria spp. (e.g. O. neglecta, O. limnetica, O. salina), Lyngbya spp. (e.g. L. majuscula, L. aestuarii), Halospirulina tapeticola, Microcystis spp., Nostoc spp., Aphanocapsa spp., and the Eukaryotes Dunaliella spp. (D. salina, D. viridis, D. parva, etc.), Dangeardinella saltitrix, Chlorella vulgaris, Navicula spp., Amphora spp. and combination thereof. Characteristics of some hypersaline microalgae are described in Table 4.

TABLE 4
List of examples of suitable Hypersaline Microalgae

| | Characteristics and | |
|---|---|---|
| Microalgae | Salinity Tolerance | Structure |
| Prokaryotes - Cyanobacteria | | |
| Aphanothece halophytica (aka | Optimally at 16-23%; | Unicellular with cell |
| Coccochloris elabens, | can be found at saturation; | wall |
| Cyanothece, Halothece) | benthic mat growth | |
| Microcoleus chthonoplastes; | Benthic mat-forming; | Sheathed filament |
| M. lyngbyaceus | salinity up to 20% and | |
| | higher; found in salterns | |
| Spirulina major; S. platensis | Found in Great Salt | Filament |
| | Lake (GSL) and | |
| | salterns; mat-forming | |
| Nodularia spumigena | Found in GSL | Filament |
| Dactylococcopsis salina | Found in salterns with | |
| | high sulfide concentrations; | |
| | 5-20% salinity | |
| Synechocystis DUN52, and | S. DUN52 up to | Unicellular |
| PCC 6803 | saturation; S. PCC 6803 | |
| | up to 8.75 to 10.5% | |
| Synechococcus PCC 7418 | | Unicellular |
| Phormidium spp. (e.g. | Found in GSL up to 18% | Filament |
| P. ambiguum, P. tenue) | | |
| Oscillatoria spp. (e.g. | Found in GSL and | Filament |
| O. neglecta, O. limnetica, | salterns; mat-forming | |
| O. salina) | | |
| Lyngbya spp. (e.g. | Found in salterns; | Filament |
| L. majuscula, L. aestuarii) | mat-forming | |
| Halospirulina tapeticola | | Coiled Filament |
| Microcystis spp. | Up to 18% | |
| Nostoc spp. | Up to 18% | |
| Aphanocapsa spp. | 6 to 36% | Small cells singly or in |
| | | pairs at low salinity; |
| | | large rounded vacuolated |
| | | cells at high salinity |
| Eukaryotes | | |
| Dunaliella spp. (D. salina, | Optimally at 7-20%; | Cell membrane; |
| D. viridis, D. parva, etc.) | up to saturation | unicellular green alga |
| Dangeardinella saltitrix | Up to saturation | Cell membrane (?); |
| | | unicellular green alga |
| Chlorella vulgaris | Hypersaline lakes, 3-25% | Unicellular with cell |
| | | wall |
| Navicula spp. | Up to 21% salinity | Diatom |
| Amphora spp. | Up to 21% salinity | Diatom |
| Isochrysis spp. | Up to 10% salinity | Cell membrane, |
| | | unicellular green alga |

In one embodiment of the processes, systems or uses the algae are selected from the group comprising or consisting of, or the algae comprise:
one or more microalgae, optionally one or more microalgal species selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., and Amphora spp.; or
genetically-engineered varieties of any of the above; or
any combinations thereof.

In one embodiment the algae or microalgae have not been genetically modified or do not originate from genetically engineered algae or microalgae.

There is increasing interest in using algal biomass or algal concentrate for a plethora of sustainable activities, such as a source of renewable energy, as a mode to safely and efficiently capture carbon dioxide from the atmosphere for carbon sequestration, and as a renewable source of chemical intermediates.

From a sustainability perspective, algal strains of commercial interest preferably do not utilize fresh water in their growth process, but use water derived from the ocean or saline aquifers to offset water losses due to evaporation from the open ponds. This constraint, based on sustainability, favors the use of marine algae that live in a saline to hypersalinity growth medium. Furthermore, the use of waste water from ponds of aquatic animals such as shrimp aquaculture ponds is even more advantageous from a sustainability perspective, because it reduces the eutrophication of the ocean or sea where the aquatic animal or shrimp effluent would have been discharged.

Suitable open ponds for algal aquaculture in the processes, systems or uses include but are not limited to those used for growing shrimp, fish, shellfish, or other types of marine organisms, or combinations thereof. Other suitable open ponds include those that are used for the production of solar salt or other minerals. Open ponds may either be lined or unlined, although the latter is typically preferred from an economical standpoint. The open ponds may be lined with plastic or bentonite or other material that is impervious to the flow of water. Pond liners constructed from various plastics may be used. Bentonite, salt, and other minerals may also be useful to reduce or minimize leakage of the growth medium into the environment. Combinations of algal aquaculture ponds of different types may offer improved performance.

Enclosed photobioreactors that are transparent so that the algae they contain can utilize the sunlight have also been proposed for the production of biofuels, and may be applicable, in special circumstances to the instant invention. These enclosed photobioreactors may comprise plastic bags, glass and plastic tubes, ponds in green-house structures, and the like. Tubular reactors were popularized by GreenFuel Technologies Corporation of Cambridge, Massachusetts for the production of biofuels, but the technology was economically unsuccessful. Plastic bag bioreactors are typified by those utilized by Algenol Biofuels of Bonita Springs, Florida. Although the capital cost of constructing a bioreactor from plastic instead of steel is substantially reduced, this type of bioreactor is still so expensive that the only commercial use is for the production of astaxanthin, a carotenoid, which is a high-value product. Thus, the use of enclosed photobioreactors is typically of commercial interest for the production of high-value products.

Open ponds are generally classified as natural, intensive, and extensive, and this type of pond is preferred for use with the instant invention. The natural open ponds are defined as those naturally occurring ponds where the conditions are right to grow algae. These ponds may contain either fresh or saline water, and they are unmanaged in terms that they lack controlled fertilizer addition and mechanical agitation. Natural open ponds that contain algae are common along the shores of the Great Salt Lake in Utah.

Both the intensive and extensive modes of aquaculture can require the controlled addition of fertilizers to the medium in order to supply the necessary nutrients, such as phosphorus, nitrogen, iron, and trace metals, that are necessary for biomass production through photosynthesis. The primary difference between the two modes of production is mixing of the growth medium. Intensive ponds employ mechanical mixing devices while extensive ponds rely on happenstance mixing. Therefore, factors that affect algae growth can be more accurately controlled in intensive aquaculture.

Intensive aquaculture ponds are frequently constructed of concrete blocks and are lined with plastic. Brine depth can generally be controlled at about 20 centimeters, which has been considered to be the optimum depth for producing algal biomass. A number of configurations of these ponds have been proposed. However, the open-air raceway ponds are typically the most important commercially. Raceway ponds employ paddle wheels to provide mixing. Chemical and biological parameters can be carefully controlled, including salt and fertilizer concentrations, pH of the brine, and purity of the culture.

Extensive aquaculture has been practiced in the hot and arid regions of Australia for the production of beta-carotene. Outdoor ponds for extensive aquaculture generally are larger than those for intensive aquaculture and normally are constructed in lake beds. The open-air ponds are typically bounded by earthen dikes. In one embodiment, no mechanical mixing devices are employed.

Algae ponds that utilize these types of aquaculture systems, others know in the art, and combinations thereof, may be used with the instant invention.

Any of a variety of products can be made from the algae, algal biomass or algal concentrate that is obtained or processed as described, and they include, but are not limited to biofuels, food, dietary supplements, nutraceuticals, cosmetics, pharmaceuticals, cosmeceuticals, wastewater treatment processes, spa products, animal feeds, human feeds, soil builders, chemicals, chemical intermediates, algal oils, proteins, carotenoids, fatty acids, lipids, specialty lipids, solar salt, and any combinations or components thereof.

In accordance with another aspect, there is provided an aquaculture system for growing algae or reducing microbes in an aqueous medium, wherein the system comprises:
a source of a process stream generated from the growth of aquatic animals; and
an algal aquaculture pond for culturing algae in fluid communication with the source of the process stream and arranged to receive the process stream therefrom, wherein the algal aquaculture pond comprises an algal aquaculture medium comprising a salinity of at least 7 wt-%, and wherein at least a portion of the algal aquaculture medium comprises the process stream,
   wherein the system further comprises a source of algal nutrients arranged for introducing algal nutrients to the process stream and/or the algal aquaculture medium.

In accordance with another aspect, there is provided an aquaculture system for growing algae or reducing microbes in an aqueous medium, wherein the system comprises:
an aquaculture pond for growing aquatic animals, wherein the aquaculture pond for growing aquatic animals comprises an aqueous medium for growing aquatic animals having a salinity of 0 to about 5 wt-%;
an algal aquaculture pond for culturing algae in fluid communication with the aquaculture pond for growing aquatic animals, wherein the algal aquaculture pond comprises an algal aquaculture medium therein having a salinity of at least about 7 wt-%,
   wherein the system further comprises a source of algal nutrients arranged for introducing algal nutrients to the process stream and/or the algal aquaculture medium.

In accordance with another aspect, there is provided an aquaculture system for carrying out any of the processes described herein. In one embodiment the system is configured to carry out the process of the present invention. In an embodiment, the system comprises:
a source of a process stream generated from the growth of aquatic animals; and
an algal aquaculture pond for culturing algae in fluid communication with the source of the process stream and arranged to receive the process stream therefrom, wherein the algal aquaculture pond comprises an algal aquaculture medium comprising a salinity of at least about 7 wt-%, and wherein at least a portion of the algal aquaculture medium comprises the process stream.

In another embodiment the system comprises an aquaculture pond for growing aquatic animals, wherein the aquaculture pond for growing aquatic animals comprises
an aqueous medium for growing aquatic animals having a salinity of 0 to about 5 wt-%; and
an algal aquaculture pond for culturing algae in fluid communication with the aquaculture pond for growing aquatic animals, wherein the algal aquaculture pond comprises an algal aquaculture medium therein having a salinity of at least about 7 wt-%.

In addition, it is appreciated that the systems disclosed herein may further include one or more of the following features:
a source of algal nutrients arranged for introducing algal nutrients to the process stream and/or the algal aquaculture medium;
means for discharging, such as an outlet conduit arranged for discharge, of at least a portion of the algal aquaculture medium to an open body of water, such as an ocean or a sea;
means for recycling, such as a recycle conduit, from the algal aquaculture pond to the source of the process stream for recycling at least a portion of the algal aquaculture medium from the algal aquaculture pond;
means for harvesting, such as an algal harvester, connected to, following, or downstream of the algal aquaculture pond;
means for recycling, such as a recycle conduit arranged to recycle, the algal concentrate from the algal harvester to the aquaculture pond for aquatic animals;
the source of the process stream comprises one or more aquaculture ponds for growing aquatic animals, and wherein the size of the aquaculture pond for growing aquatic animals is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or wherein the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares;
the source of the process stream comprises an open aquaculture pond for the growth of aquatic animals;
the aquaculture ponds for the growth of the aquatic animals and/or the algal aquaculture ponds are open ponds;
the salinity of the algal aquaculture medium is at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%;
the process stream comprises a salinity of from 0 to about 5 wt-%;
the process stream comprises a waste stream or a recycle stream generated from the growth of aquatic animals;
the aquatic animals are selected from the group consisting of crustaceans, shrimps, fishes, molluscs, shellfishes, and any combination thereof, or the aquatic animals are selected from the group consisting of Penaeid family shrimps, Penaeus chinensis, P. monodon, P. japonicus, P. merguinsis, P. penicillatus, Metapenaeus ensis, and P. vannamei;
the process stream comprises:
   one or more of the viruses selected from the group consisting of Monodon baculovirus, Baculoviral midgut gland necrosis virus, White spot syndrome virus, Infectious hypodermal and haematopoietic necrosis virus, Hepatopancreatic parvovirus, Yellow head virus, Taura syndrome virus, Infectious myonecrosis virus, Macrobrachium rosenbergii nodavirus (White Tail Disease), Laem-Singh virus, and Mourilyan virus; and/or
   one or more bacteria contributing to one or more selected from the group consisting of Blackshell Disease, Septic Hepatopancreatic Necrosis, Tail Rot, Brown Gill Disease, Swollen Hindgut Syndrome, Firefly Disease, Luminous Bacterial Disease, Texas Necrotizing Hepatopancreatitis (TNHP), Granulamatous hepatopancreatitis, Texas Pond Mortality Syndrome (TPMS), Peru Necrotizing Hepatopancreatitis (PNHP), Mycobacterium Infection, Zoea II Syndrome, Shrimp Tuberculosis, Rickettsial infection, and combinations thereof; and/or
the process stream comprises algal nutrients therein for the culturing of the algae.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. That said, it is understood that any one or more features disclosed herein may be combined.

As used herein, a plurality of items, structural elements, compositional elements, and/or materials may be presented in a common list for convenience. However, these lists should be construed as though each member of the list is individually identified as a separate and unique member. Thus, no individual member of such a list should be construed as a de facto equivalent of any other member of the same list solely based on their presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

### EXAMPLES

### Example 1

Penaeus monodon were grown in an open pond using traditional aquaculture methods and were suffering from Acute Hepatopancreatin Necrosis Disease (AHPND), causative agent of Vibrio parahaemolyticus with a common name of “Early Mortality Syndrome” (EMS). The salinity of the aqueous media in the shrimp pond was 4 wt-%. The waste stream from that pond was discharged to an algal aquaculture pond in which the algae Dunaliella salina were grown. The algae were grown in open ponds in which the algal aquaculture medium had a salinity of 20 wt-%. The waste stream entering the algal aquaculture pond was first treated with nutrients for algal growth, including primarily nitrogen and phosphorus with lesser amounts of iron, manganese, copper, and zinc. The salinity of the waste stream was also increased to match the salinity of the algal aquaculture medium when the nutrients were added. The treated waste stream was then introduced into the algal aquaculture ponds. The population of Vibrio parahaemolyticus in the waste stream was measured by cell count. The residence time in the algal aquaculture ponds was approximately 10 days. The population of Vibrio parahaemolyticus in the algal aquaculture medium discharged from the algal aquaculture pond was measured by cell count. The treatment of the waste stream in the algal aquaculture pond resulted in a 75% reduction in the population of Vibrio parahaemolyticus, with the population of Vibrio parahaemolyticus in the discharged algal aquaculture medium being lower than that of local ocean water.

### Example 2

A marine sample was collected from Pond 1 in a traditional shrimp aquaculture farm located in Sonora, Mexico. The sample comprised wet, black mud from the bottom sediments of the shrimp pond that contained organic matter. The 20 liter sample was collected and immediately placed on ice until subsamples of the marine sample were withdrawn the following day. Test tubes were prepared with salinity increments of 0, 2, 8, 12, 18, and 25 wt-% NaCl by using a saturated solution of marine brine and diluting with fresh water. A total of nine milliliters of each of these salinity increments were placed in a test tube and 1.0 grams of the sample was added to each test tube at the different salinity increments. The test tubes were placed in an orbital incubator operating at 150 rpm for 24 hours and at 30 degrees Celsius. Thereafter, 100 microliter samples were seeded onto Thiosulfate Citrate Bile Sucrose agar plates (TCBS) by extension, and incubated for 24 hours at 30 degrees Celsius. After this incubation period, the Colony Forming Unit (CFUs) per gram, or CFU's/gram, were counted. The Vibrio bacteria incubated on TCBS agar produced either yellow or green colonies, depending if they could ferment sucrose, or not. When sucrose fermentation occurs, yellow colonies are produced by species, such as Vibrio cholera. Vibrio species known to be shrimp pathogens, such as Vibrio parahaemolyticus, produce green colonies when incubated on TCBS agar. In all of the colonies observed, about 95% of the colonies during analysis were yellow, and about 5% of the colonies were green. The kill rate at the different salinities was computed based on the CFU/gram count counted at 2 wt-% NaCl. The kill rate was computed as: (1−(CFU/gram at the salinity of interest divided by the CFU/gram at 2 wt-% NaCl))×100%. At 8 and 12 wt-% NaCl, the percentage reduction in CFU's/gram were 91.3 and 98.9%, respectively. No CFU's/gram were observed at 18 and 25 wt-% NaCl-thus the percentage reduction in CFU's/gram were essentially 100%, within measurement accuracy.

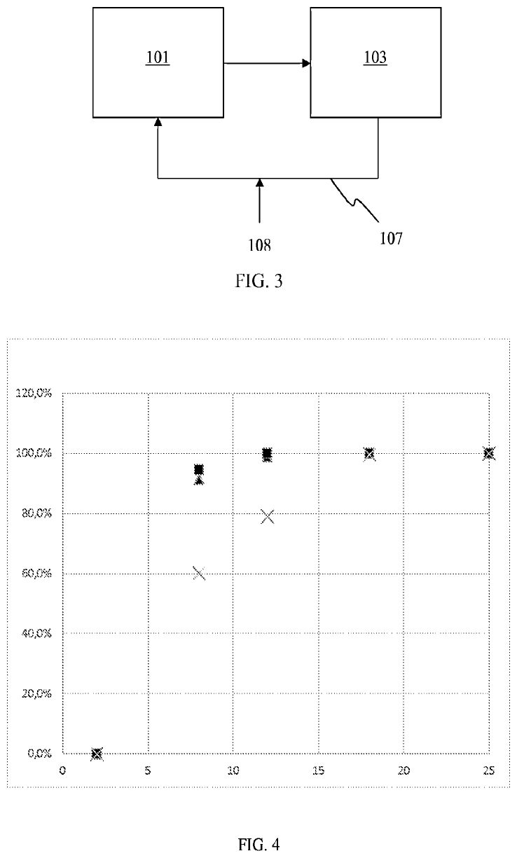

A graphical representation of the results is shown in FIG. 4 with triangles, the treatment salinity (wt-% NaCl) on the x-axis and counts/counts at 2 wt-% NaCl on the y-axis. Excellent reduction of Vibrio species was shown already at 8 wt-% NaCl and essentially no Vibrio species were found at salinities at or above 12 wt-% NaCl after treatment for 24 hours.

### Example 3

A marine sediment sample was collected from Pond 2 in a traditional shrimp aquaculture farm located in Sonora, Mexico. The sample comprised wet, black mud from the bottom sediments of the shrimp pond that contained organic matter. The 20 liter sample was collected and immediately placed on ice until subsamples of the marine sample were withdrawn the following day. Test tubes were prepared with salinity increments of 0, 2, 8, 12, 18, and 25 wt-% NaCl by using a saturated solution of marine brine and diluting with fresh water. A total of nine milliliters of each of these salinity increments were placed in a test tube and 1.0 grams of sediment was added to each test tube at the different salinity increments. The test tubes were placed in an orbital incubator operating at 150 rpm for 24 hours and at 30 degrees Celsius. Thereafter, 100 microliter samples were seeded onto Thiosulfate Citrate Bile Sucrose agar plates (TCBS) by extension, and incubated for 24 hours at 30 degrees Celsius. After this incubation period, the Colony Forming Unit (CFUs) per gram, or CFU's/gram, were counted. The Vibrio bacteria incubated on TCBS agar produce either yellow or green colonies, depending if they can ferment sucrose, or not. When sucrose fermentation occurs, yellow colonies are produced by species, such as Vibrio cholera. Vibrio species known to be shrimp pathogens such as Vibrio parahaemolyticus, produce green colonies when incubated on TCBS agar. In all of the colonies observed, about 95% of the colonies during analysis were yellow, and about 5% of the colonies were green. The kill rate at the different salinities was computed based on the CFU/gram count counted at 2 wt-% NaCl. The kill rate was computed as: (1−(CFU/gram at the salinity of interest divided by the CFU/gram at 2 wt-% NaCl))×100%. At 8 wt-% NaCl, the percentage reduction in CFU's/gram was 94.4%. No CFU's/gram were observed at 12, 18, and 25 wt-% NaCl-thus the percentage reduction in CFU's/gram were essentially 100%, within measurement accuracy at these salinities.

A graphical representation of the results is shown in FIG. 4 with squares, the treatment salinity (wt-% NaCl) on the x-axis and counts/counts at 2 wt-% NaCl on the y-axis. Excellent reduction of Vibrio species was shown already at 8 wt-% NaCl and essentially no Vibrio species were found at salinities at or above 12 wt-% NaCl after treatment for 24 hours.

### Example 4

A marine sediment sample was collected from Pond 3 in a traditional shrimp aquaculture farm located in Sonora, Mexico. The sample comprised wet, black mud from the bottom sediments of the shrimp pond that contained organic matter. The 20 liter sample was collected and immediately placed on ice until subsamples of the marine sample were withdrawn the following day. Test tubes were prepared with salinity increments of 0, 2, 8, 12, 18, and 25 wt-% NaCl by using a saturated solution of marine brine and diluting with fresh water. A total of nine milliliters of each of these salinity increments were placed in a test tube and 1.0 grams of sediment was added to each test tube at the different salinity increments. The test tubes were placed in an orbital incubator operating at 150 rpm for 24 hours and at 30 degrees Celsius. Thereafter, 100 microliter samples were seeded onto Thiosulfate Citrate Bile Sucrose agar plates (TCBS) by extension, and incubated for 24 hours at 30 degrees Celsius. After this incubation period, the Colony Forming Unit (CFUs) per gram, or CFU's/gram, were counted. The Vibrio bacteria incubated on TCBS agar produce either yellow or green colonies, depending if they can ferment sucrose, or not. When sucrose fermentation occurs, yellow colonies are produced by species such as Vibrio cholera. Vibrio species known to be shrimp pathogens such as Vibrio parahaemolyticus, produce green colonies when incubated on TCBS agar. In all of the colonies observed, about 95% of the colonies during analysis were yellow, and about 5% of the colonies were green. The kill rate at the different salinities was computed based on the CFU/gram count counted at 2 wt-% NaCl. The kill rate was computed as: (1−(CFU/gram at the salinity of interest divided by the CFU/gram at 2 wt-% NaCl))×100%. At 8, 12, and 18 wt-% NaCl, the percentage reduction in CFU's/gram was 60.2, 79.0, and 99.7%, respectively. No CFU's/gram were observed at 25 wt-% NaCl-thus the percentage reduction in CFU's/gram were essentially 100%, within measurement accuracy at these salinities.

A graphical representation of the results is shown in FIG. 4 with crosses, the treatment salinity (wt-% NaCl) on the x-axis and counts/counts at 2 wt-% NaCl on the y-axis. Excellent reduction of Vibrio species was shown already at 8 wt-% NaCl and essentially no Vibrio species were found at salinities at or above 18 wt-% NaCl after treatment for 24 hours.

### REFERENCES CITED

### U.S. Patent Documents

U.S. Pat. No. 5,692,455 Fluidized bed production of oysters and other filter feeding bivalve mollusks using shrimp pond water, Wang; Jaw Kai (Honolulu HI)
U.S. Pat. No. 5,732,654 Open air mariculture system and method of culturing marine animals, Perez; Carlos E. (Guayaquil, EC), Hunter; Max L. (Guayaquil, EC)
U.S. Pat. No. 6,440,466 Composition for treating white spot syndrome virus (WSSV) infected tiger shrimp Penaeus monodon and a process for preparation thereof, Desai; Ulhas Manohar (Goa, IN), Achuthankutty; Chittur Thelakkat (Goa, IN), Sreepada; Rayadurga Anantha (Goa, IN)
U.S. Pat. No. 6,518,317 Antiviral Agents, Sagawa, H. et al. (Otsu, JP)
U.S. Pat. No. 6,615,767 Aquaculture method and system for producing aquatic species, Untermeyer; Thomas C. (Lakehills, TX), Williams; Bill G. (Waco, TX), Easterling; Gerald (Carrollton, TX)
U.S. Pat. No. 7,140,323 Reducing the level of bacteria and viruses in aquaculture, Ashworth; David Wilson (Lowdham, GB), Benz; Angelika (Romerberg, DE), Zeller; Dieter (Speyer, DE)
U.S. Pat. No. 7,306,818 Method for preventing and/or treating viral infection in aquatic animals, Su; Wei-Chih (Tainan County, TW), Hsiao; Hsiung (Tainan County, TW), Yang; Chiung-Hua (Tainan County, TW)
U.S. Pat. No. 7,652,050 Methods for increasing the survival of aquatic animals infected with an aquatic virus, Sandino; Ana Maria (Santiago, CL), Mlynarz; Geraldine Z. (Santiago, CL)

### Other Publications

Walker Peter J., Winton James R., “Emerging viral diseases of fish and shrimp” 1 CSIRO Livestock Industries, Australian Animal Health Laboratory (AAHL), 5 Portarlington Road, Geelong, Victoria, Australia 2 USGS Western Fisheries Research Center, 6505 NE 65^{th} Street, Seattle, Washington, USA (2010)
Boyd, Claude E. and Musig, Yont, “Shrimp Pond Effluents: Observations of the Nature of the Problem on Commercial Farms”, 1992 Proceedings of the Special Session on Shrimp Farming, World Aquaculture Society, Baton Rouge, LA Wyban, J., Editor
Wijffels, Rene H. and Barbosa, Maria J., “An Outlook on Microalgal Biofuels”, Science, Vol 329, Aug. 13, 2010 pp 796-799
Granada et al. 2015, Reviews in Aquaculture, “Is integrated multitrophic aquaculture the solution to the sectors' major challenges?—a review” (2015) 6, 1-18
Walker Peter J., Winton James R., “Emerging Viral Diseases of Fish and Shrimp”, Vet. Res. (2010) 41:51
Gill, Tom A., “Waste from Processing Aquatic Animals and Animal Products: Implications on aquatic pathogen transfer”, Series Title: Fisheries Circular C956, 2000, 26 pg, X9199/E (2000)
Johannes, R. E. and Satomi, Masako, “Composition and Nutritive Value of Fecal Pellets of a Marine Crustacean”, Limnol. Oceanogr. 11:191-197 (1966)
Vaiphasa, C., de Boer, W. F., Skidmore, A. K., Panitchart, S., Vaiphasa, T., Bamrongrugsa, N., Santitamnont, P., “Impact of solid shrimp pond waste materials on mangrove growth and mortality: a case study from Pak Phanang, Thailand”, Hydrobiologia (2007) 591:47-57.
Harry, L. and Rabanal, Herminio, Editors, South China Sea Fisheries Development Coordinating Programme, “Manual on Pond Culture of Penaeid Shrimp, (1978)
Houston, J. E. and Nieto, Amelia “Regional Shrimp Market Responses to Domestic Landings and Imports,” Journal of Food Distribution Research, pp, 99-107, February 1988.
Fegan, Second FAO Symposium on AHPND Bangkok, 2016
Loc Tran. Second FAO Symposium on AHPND Bangkok 2016

1. A process for culturing algae and/or reducing pathogenic microbes from an aqueous medium, wherein the process comprises:
growing aquatic animals in an aquaculture pond comprising an aqueous medium comprising a salinity of 0 to 5 wt-%;
feeding a process stream generated from the growth of aquatic animals to an algal aquaculture pond; and
culturing algae in the algal aquaculture pond in an algal aquaculture medium comprising a salinity of at least 7 wt-%, wherein at least a portion of the algal aquaculture medium comprises the process stream.

2. The process of claim 1, comprising feeding the process stream to the algal aquaculture pond from more than one aquaculture ponds for the growth of the aquatic animals.

3. The process of claim 1, wherein the process further comprises:
feeding the algal aquaculture medium from the algal aquaculture pond to one or more further aquaculture ponds for the growth of algal aquacultures or aquatic animals; and/or
recycling at least a portion of the algal aquaculture medium from the algal aquaculture pond back to a pond for the culture of aquatic animals and combining the algal aquaculture medium with an aqueous medium that reduces a salinity of the algal aquaculture medium.

4. The process of claim 1, further comprising discharging at least a portion of the algal aquaculture medium after the culturing of algae to open water, such as an ocean or a sea.

5. The process of claim 1, further comprising adding algal nutrients to the process stream for the culturing of the algae.

6. The process of claim 1, further comprising preparing a product, optionally a feed product, from an algal concentrate obtained by harvesting algae from the algal aquaculture pond.

7. The process of claim 1, wherein the salinity of the process stream is increased in a conduit between a growth pond within which the growth of aquatic animals takes place and the algal aquaculture pond, optionally by adding solid salt materials or an aqueous medium having a higher salinity than that of the process stream.

8. (canceled)

9. An aquaculture system for growing algae or reducing microbes in an aqueous medium, wherein the system comprises:
a source of a process stream generated from the growth of aquatic animals in an aqueous medium, comprising a salinity of from 0 to 5 wt-%;
an algal aquaculture pond for culturing algae in fluid communication with the source of the process stream and arranged to receive the process stream therefrom, wherein the algal aquaculture pond comprises an algal aquaculture medium comprising a salinity of at least 7 wt-%, and wherein at least a portion of the algal aquaculture medium comprises the process stream; and
means for increasing salinity of the algal aquaculture medium and/or salinity of the process stream.

10. The system of claim 9, wherein the system further comprises a source of algal nutrients arranged for introducing algal nutrients to the process stream and/or the algal aquaculture medium.

11. The system of claim 9, wherein the system further comprises a recycle conduit from the algal aquaculture pond to the source of the process stream for recycling at least a portion of the algal aquaculture medium from the algal aquaculture pond.

12. The system of claim 9, wherein the source of the process stream comprises an aquaculture pond for growing aquatic animals, and wherein the size of the aquaculture pond for growing aquatic animals is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or wherein the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares; or
the process of anyone of claims 1 to 10, wherein the size of a pond for the growth of the aquatic animals is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or wherein the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares, and/or wherein the size of the algal aquaculture pond is about 0.1-about 1000 hectares, about 0.1-about 200 hectares, about 0.1-about 100 hectares, about 0.1-about 20 hectares, about 1-about 50 hectares, about 1-about 20 hectares, about 1-about 10 hectares, or about 5-about 10 hectares.

13. The system of claim 9, wherein the system is configured to carry out the process.

14. The system of claim 9, wherein the salinity of the algal aquaculture medium is at least about 8 wt-%, at least about 9 wt-%, at least about 10 wt-%, at least about 11 wt-%, at least about 12 wt-%, at least about 13 wt-%, at least about 14 wt-%, at least about 15 wt-%, at least about 16 wt-%, at least about 17 wt-%, at least about 18 wt-%, at least about 19 wt-%, at least about 20 wt-%, at least about 21 wt-%, at least about 22 wt-%, at least about 23 wt-%, at least about 24 wt-%, or at least about 25 wt-%.

15. The system of claim 9, wherein the process stream comprises a salinity of from 0 to about 5 wt-%.

16. The system of claim 9, wherein the aquatic animals are selected from the group consisting of crustaceans, shrimps, fishes, molluscs, shellfishes, and any combination thereof, or the aquatic animals are selected from the group consisting of Penacid family shrimps, Penaeus chinensis, P. monodon, P. japonicus, P. merguinsis, P. penicillatus, Metapenaeus ensis, P. vannamei and Litopenaeus vannamei.

17. The system of claim 9, wherein the process stream comprises:
one or more of the viruses selected from the group consisting of Monodon baculovirus, Baculoviral midgut gland necrosis virus, White spot syndrome virus, Infectious hypodermal and haematopoietic necrosis virus, Hepatopancreatic parvovirus, Yellow head virus, Taura syndrome virus, Infectious myonecrosis virus, Macrobrachium rosenbergii nodavirus (White Tail Disease), Laem-Singh virus, and Mourilyan virus; and/or
one or more bacteria contributing to one or more selected from the group consisting of Blackshell Disease, Septic Hepatopancreatic Necrosis, Tail Rot, Brown Gill Disease, Swollen Hindgut Syndrome, Firefly Disease, Luminous Bacterial Disease, Texas Necrotizing Hepatopancreatitis (TNHP), Granulamatous hepatopancreatitis, Texas Pond Mortality Syndrome (TPMS), Peru Necrotizing Hepatopancreatitis (PNHP), Mycobacterium Infection, Zoca II Syndrome, Shrimp Tuberculosis, Rickettsial infection, and combinations thereof.

18. The system of claim 9, wherein the algae comprises or is selected from
one or more microalgal species selected from the group consisting of Amphora sp., Anabaena sp., Anabaena flos-aquae, Ankistrodesmus falcatus, Arthrospira sp., Arthrospira (Spirulina) obliquus, Arthrospira (Spirulina) platensis, Botryococcus braunii, Ceramium sp., Chaetoceros gracilis, Chlamydomonas sp., Chlamydomonas mexicana, Chlamydomonas reinhardtii, Chlorella sp., Chlorella fusca, Chlorella protothecoides, Chlorella pyrenoidosa, Chlorella stigmataphora, Chlorella vulgaris, Chlorella zofingiensis, Chlorococcum citriforme, Chlorococcum littorale, Closterium sp., Coccolithus huxleyi, Cosmarium sp., Crypthecoddinium cohnii, Cryptomonas sp., Cyclotella cryptica, Cyclotella nana, Dunaliella sp., Dunaliella bardawil, Dunaliella salina, Dunaliella tertiolecta, Dunaliella viridis, Euglena gracilis, Fragilaria, Fragilaria sublinearis, Gracilaria, Haematococcus pluvialis, Hantzschia, Isochrysis galbana, Microcystis sp., Monochrysis lutheri, Muriellopsis sp., Nannochloris sp., Nannochloropsis sp., Nannochloropsis salina, Navicula sp., Navicula saprophila, Neochloris oleoabundans, Neospongiococcum gelatinosum, Nitzschia laevis, Nitzschia alba, Nitzschia communis, Nitzschia paleacea, Nitzschia closterium, Nitzschia palea, Nostoc commune, Nostoc flagellaforme, Pavlova gyrens, Peridinium, Phaeodactylum tricornutum, Pleurochrysis carterae, Porphyra sp., Porphyridium aerugineum, Porphyridium cruentum, Prymnesium, Prymnesium paruum, Pseudochoricystis ellipsoidea, Rhodomonas sp., Scenedesmus sp., Scenedesmus braziliensis, Scenedesmus obliquus, Scenedesmus quadricauda, Scenedesmus acutus, Scenedesmus dimorphus, Schizochytrium sp., Scytonema, Skeletonema costatum, Spirogyra, Schiochytrium limacinum, Stichococcus bacillaris, Synechoccus, Tetraselmis sp., Tolypothrix sp., genetically-engineered varieties thereof, and any combinations thereof; or
one or more prokaryotes selected from the group consisting of Aphanothece halophytica, Microcoleus chthonoplastes, M. lyngbyaceus, Spirulina major, S. platensis, Nodularia spumigena, Dactylococcopsis salina, Synechocystis DUN52, PCC 6803, Synechococcus PCC 7418, Phormidium spp., Oscillatoria spp., Lyngbya spp., Halospirulina tapeticola, Microcystis spp., Nostoc spp., and Aphanocapsa spp.; or
one or more eukaryotes selected from the group consisting of Dunaliella spp., Dangeardinella saltitrix, Chlorella vulgaris, Navicula spp., and Amphora spp.; or
genetically-engineered varieties of any of the above; or
any combinations thereof.

19. Use of the system of claim 9 for culturing algae and/or for reducing pathogenic microbes from a process stream generated from the growth of aquatic animals.
